(19) United States
(12) Patent Application Publication
(10) Pub. No.: US 2013/0211259 A1
(43) Pub. Date: Aug. 15, 2013

(54) DETERMINATION OF JOINT CONDITION BASED ON VIBRATION ANALYSIS

(71) Applicant: JOINTVUE, LLC, (US)

(72) Inventors: Richard D. Komistek, Raleigh, NC (US); Sumesh Mahesh Zingde, Alpharetta, GA (US)

(73) Assignee: JOINTVUE, LLC, Columbus, OH (US)

(21) Appl. No.: 13/841,632

(22) Filed: Mar. 15, 2013

Related U.S. Application Data
(63) Continuation-in-part of application No. 13/196,701, filed on Aug. 2, 2011.
Application No. 13/196,701 is a continuation-in-part of application No. PCT/US2010/022939, filed on Feb. 2, 2010.
Application No. PCT/US2010/022939 is a continuation of application No. 12/364,267, filed on Feb. 2, 2009, now Pat. No. 8,444,564.

(51) Int. Cl. A61B 8/08; A61B 8/00

(52) U.S. Cl. CPC A61B 8/5223; A61B 8/0875; A61B 8/463; A61B 8/466
USPC 600/440; 600/453

(57) ABSTRACT

Methods and a system of determining a condition of a joint. A first signal indicative of a vibration generated by motion of the joint is received in a processor. The processor generate a vibroarthrograph from the first signal and extract a first signal feature from the vibroarthrograph based on a first statistical parameter of the vibroarthrograph. The first signal feature is then compared to a plurality of signal features in a database, each of the plurality of signal features in the database being associated with at least one joint condition. A condition of the joint may then be determine based at least in part on a correspondence between the first signal feature and a signal feature of the plurality of signal features in the database. Multiple signal features may also be combined into one or more functions that provide separation between vibrations from healthy joints and vibrations from injured joints.

This application is a continuation-in-part of U.S. patent application Ser. No. 13/196,701, filed Aug. 2, 2011, which claims the filing benefit of PCT Patent Application No. PCT/US2010/022939, filed on Feb. 2, 2010, and is a continuation-in-part of U.S. patent application Ser. No. 12/364,267, filed on Feb. 2, 2009, the disclosures of which are all incorporated by reference herein in their entireties.

### FIELD OF INVENTION

The present invention relates generally to systems and methods for characterizing a joint defect based on detected vibrations and acoustic signatures, and more specifically, to assisting pre-operative diagnosis, intra-operative implantation techniques, and post-operative evaluation of native, injured, arthritic, and artificial joints using vibroarthrography.

### BACKGROUND

Joint injuries are one of the most commonly reported musculoskeletal problems. These injuries can occur due to various reasons. In young adults, sports are a major cause of injuries. These injuries tend to be mainly involve the soft tissue structures of the joint (e.g., meniscus and cruciate ligament injuries in the knee joint and labral injuries in the hip joint). In older subjects, arthritic degeneration (such as rheumatoid or osteoarthritis) of joints such as knees and hips is a common phenomenon, and may result from a variety of traumatic causes. According to the Arthritis Foundation, arthritis-related problems are second only to heart disease as the leading cause of work disability. Mechanical loading, especially dynamic loading, is believed to play a major role in the degenerative process. This loading may result in bone to bone contact where the cushioning layers are damaged, thereby causing pain for the patient. Osteoarthritis in particular can be extremely disabling, leading to discomfort and often excruciating pain.

Depending on the type and nature of the joint damage, different treatment modalities can be pursued. For soft-tissue damage, mainly meniscul and ligament injuries, arthroscopic procedures can be implemented to determine the nature of the injury as well as repair the damage caused by it. More chronic or severe joint damage, such as that caused by osteoarthritis, is typically treated in a stepwise treatment regime which includes pain relievers, NSAIDS, and joint visco supplementation. If these treatment methods fail, they may be followed as a last resort with artificial orthopedic implants, which are designed to replace the damaged articulating surfaces of the injured joint and thereby provide pain relief. Joint implants may allow a subject with severe osteoarthritis to return to a normal daily life. One exemplary type of joint implant procedure is known as a total knee arthroplasty.

Multiple artificial joint designs exist that seek to duplicate the geometry and behavior of a healthy knee joint. The differences in these designs are based on factors such as condylar geometry, bearing mobility, ligament preservation vs. substitution, and fixation methods.

Irrespective of the type of injury sustained by an individual, the treatment modality typically includes three phases: (1) pre-operative diagnosis of the joint and selection of a treatment regime; (2) implementation of the regime by non-invasive physiotherapy and stabilization techniques or invasive surgery (e.g., implantation of the replacement joint); and (3) post operative evaluation of the joint.

One of the major problems in determining knee joint conditions caused by soft tissue damage or arthritis is the ability to detect the cause of abnormal joint conditions early. Use of X-rays, computer assisted tomography (CAT) and magnetic resonance imaging (MRI) scans are limited to providing information on defects that are gross in nature. In addition, in the case of artificial joints, implants may include metal parts, thus MRI scans typically cannot be used post-operatively in implanted patients. Arthroscopic procedures can be used to overcome the deficiencies in available imaging techniques. However, arthroscopic procedures are semi-invasive and thus undesirable from a pre-operative diagnostic stand point due to the need for surgery and the corresponding expense and patient discomfort.

Therefore, there is a need for improved methods and systems for determining the condition of joints and the effectiveness of implemented treatment modalities without the use of ionizing radiation or invasive procedures, and that are adaptable for use during preoperative, perioperative, and postoperative phases of the joint replacement process.

### BRIEF DESCRIPTION OF THE DRAWINGS

The accompanying drawings, which are incorporated in and constitute a part of this specification, illustrate embodiments of the invention and, together with a general description of the invention given below, serve to explain the principles of the invention.

FIG. 1 is high level view of a process for determining the condition of a joint.

FIG. 2 is perspective view of a knee of a patient including a joint monitoring apparatus in the form of a knee brace.

FIG. 3 is a schematic view of a joint monitoring apparatus of FIG. 2.

FIG. 4 is a schematic view of an exemplary computing environment for use with the joint monitoring apparatus of FIG. 3.

FIG. 5 is a diagrammatic view of a joint diagnostic system that may be hosted by the computing environment of FIG. 4.

FIG. 6 is a diagrammatic view of an evaluation sheet that may be used to gather data in conjunction with the diagnostic system of FIG. 5.

FIG. 7 is a perspective view of a knee joint showing an exemplary positioning of accelerometers for gathering vibration data.

FIG. 8 is a graphical view of an exemplary signal received from one of the accelerometers in FIG. 7 including a low-pass filtered portion and a high-pass filtered portion of the received signal.

FIGS. 9A-9C are schematic views of filters and spectral analysis techniques for separating the low-pass and high-pass filtered portions of the signal in FIG. 8.

FIG. 10 is a graphical view of exemplary vibrations received from a healthy joint and an injured joint.

FIG. 11 is a diagrammatic view of a vibration pattern classifier that may be implemented in the diagnostic system of FIG. 5.

FIGS. 12A-12E are graphical views illustrating results of statistically separating healthy joints from injured joints based on statistical features of vibroarthrograms.

FIG. 13 is a schematic view of method of analyzing captured time domain signal using a Fourier transform and short-time Fourier transform.

FIG. 14A is a graphical view of a time domain signal of a joint vibration.

FIG. 14B is a graphical view of a short-time Fourier transformed version of the time domain signal in FIG. 14A.

FIG. 15 is a diagrammatic view of a display that may be provided by the diagnostic system of FIG. 5 including an image of a 3-D model of the joint, images showing contact areas between bones comprising the joint, and a vibroarthrograph of the vibrations generated by the joint.

### SUMMARY

In an embodiment of the invention, a method of determining a condition of a joint is provided. The method includes receiving a first signal indicative of a vibration generated by motion of the joint in a processor. The method further includes generating a vibroarthrograph from the first signal and extracting a first signal feature from the vibroarthrograph based on a first statistical parameter of the vibroarthrograph. The first signal feature is compared to a plurality of signal features in a database, each of the plurality of signal features in the database being associated with at least one joint condition. The method further includes determining the condition of the joint based at least in part on a correspondence between the first signal feature and a signal feature of the plurality of signal features in the database.

In another embodiment of the invention, an additional method of determining a condition of a joint is provided. The method includes receiving a first signal indicative of a vibration generated by motion of the joint in the processor and generating a vibroarthrograph from the first signal. The method further includes receiving a second signal indicative of a position of the joint during the motion of the joint in the processor, and determining an orientation of a 3-D model of the joint based at least in part on the second signal. The method synchronizes the first and second signals so that each point on the vibroarthrograph is associated with a position of the joint, and displays a first image representing the orientation of the 3-D model of the joint, and a second image representing the vibroarthrograph. The first and second images are synchronized so that movement of the 3-D model corresponds to a position of a sample window in the vibroarthrograph.

In yet another embodiment of the invention, another method of determining a condition of the joint is provided. The method includes receiving a first signal indicative of a vibration generated by a motion of the joint in the processor, generating a vibroarthrograph based on the first signal, and extracting a plurality of signal features from the vibroarthrograph, with each signal feature being based on a different statistical parameter. The method further includes defining a plurality of feature vectors of the vibroarthrograph, each feature vector being based on one or more weighted signal features of the plurality of signal features and being associated with at least one joint condition, and determining a score for each of the plurality of feature vectors based on the vibroarthrograph. The method further includes diagnosing the joint by selecting a joint condition associated with the feature vector having the highest score.

In yet another embodiment of the invention, a system for determining a condition of the joint is provided. The system includes a processor and a memory including program code. When executed by the processor, the program code causes the processor to receive a first signal indicative of a vibration generated by a motion of the joint, generate a vibroarthrograph from the first signal, and extract a first signal feature from the vibroarthrograph based on a first statistical parameter of the vibroarthrograph. The code may further cause the processor to compare the first signal feature to a plurality of signal features in a database, each of the plurality of signal features in the database being associated with at least one joint condition, and determine the condition of the joint based at least in part on a correspondence between the first signal feature and a signal feature of the plurality of signal features in the database.

In yet another embodiment of the invention, another system for determining a condition of a joint is provided. The system includes a processor and a memory including program code. When executed by the processor, the code causes the processor to receive a first signal indicative of a vibration generated by a motion of the joint, generate a vibroarthrograph based on the first signal, and extract a plurality of signal features from the vibroarthrograph, each signal feature being based on a different statistical parameter. The code further causes the processor to define a plurality of feature vectors of the vibroarthrograph, each feature vector being based on one or more weighted signal features of the plurality of signal features and being associated with at least one joint condition, determine a score for each of the plurality of feature vectors based on the vibroarthrograph, and diagnose the joint by selecting a joint condition associated with the feature vector having the highest score.

### DETAILED DESCRIPTION

The present invention addresses the foregoing problems and other shortcomings, drawbacks, and challenges of determining a condition of a joint. The methods and systems described herein may be used preoperatively to diagnose defects within the joint, perioperatively to adjust an artificial joint or repair soft tissue structures, and postoperatively to diagnose and monitor the functions of the surgical procedures such as joint wear.

Vibration and acoustic analysis of joints is based on the principle that joints are functionally controlled by a mechanical system governed by three unique types of forces. These forces are: (1) active forces resulting from motion, such as those resulting from a muscle flexing or relaxing; (2) constraining forces that constrain motion, such as those resulting from ligaments being in tension; and (3) interaction forces that resist motion, such as those acting upon bones. In addition to these three types of forces, the soft tissue in the joint (e.g., the cartilage and the meniscus in a knee) produce a dampening effect distributing the compressive loads acting on the joint.

It has been determined that an injury or defect to any one of the joint ligaments or other soft-tissue structures may result in detectable vibrations and/or an acoustic pattern representative of the type of joint injury and/or the severity of the injury. These auditory and vibrational changes are produced as the bones move in a distorted kinematic pattern and produce vibration and acoustic signals when interacting with the defective/injured body structures. Thus, the kinematics, acoustic signature and vibrations of injured joints may differ significantly from the look and vibration content of a properly balanced joint moving through the same range and types of motion. Moreover, kinematic patterns that are non-optimal due to a poorly fitted joint implant, or an implant that has experienced significant wear, may alter the vibrations and acoustics produced by the joint.

Although embodiments of the invention are generally described herein with respect to a knee joint for the sake of simplicity, those skilled in the art will recognize that the methods and systems described may also be used for diagnosing and treating other types of joints without departing from the scope of embodiments of the invention. Moreover, embodiments of the invention may apply to methods and systems used for the condition of joints in a veterinary setting on non-human subjects, such as dogs, cats, race horses, farm and zoo animals, or any other animal undergoing joint evaluation and/or treatment.

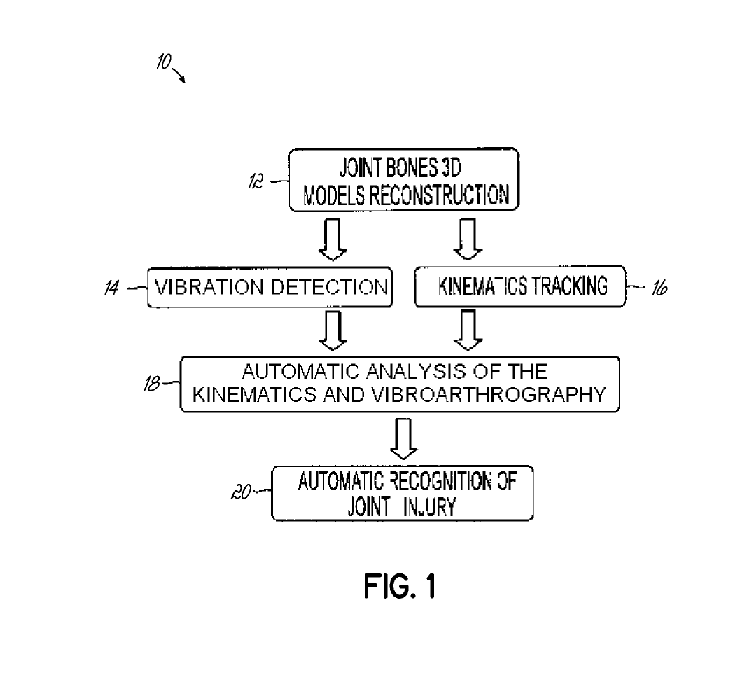

Referring now to FIG. 1, a high level overview of an exemplary method 10 for determining a joint condition or type of joint injury in accordance with an embodiment of the invention is presented. In block 12, a 3-D model of the joint is constructed. This 3-D model may be a patient specific model, and may be generated by obtaining a plurality of raw RF signals using pulse echo ultrasound acquisition methodologies. A bone contour may then be isolated in each of the plurality of RF signals and transformed into a point cloud representing the joint. The point clouds may then be used to optimize a 3-D model of the bone such that the patient-specific model may be generated. Methods of generating 3-D joint models and re-constructing joint cartilage are described in U.S. patent application Ser. No. 13/758,151 filed on Feb. 4, 2013 and entitled “METHOD AND APPARATUS FOR THREE DIMENSIONAL RECONSTRUCTION OF A JOINT USING ULTRASOUND”, the disclosure of which is incorporated herein by reference in its entirety.

In block 14, acoustic vibrations are detected as the joint is moved. These vibrations may be detected using a suitable transducer, such as one or more accelerometers coupled to the patient in proximity to the joint. As used herein, the term “vibration” is intended to encompass any oscillatory, periodic, or random motion of particles of an elastic body or medium. “Vibrations” thus include mechanical vibrations (such as those that may be produced by a moving joint), acoustic energy (i.e., the sound produced by the joint), or any other type of time varying phenomena by which kinetic energy propagates through a medium that is detectable by an accelerometer or other mechanical to electrical energy transducer. The signals generated by the transducers may be transmitted to a computer either through a wired connection, or wirelessly.

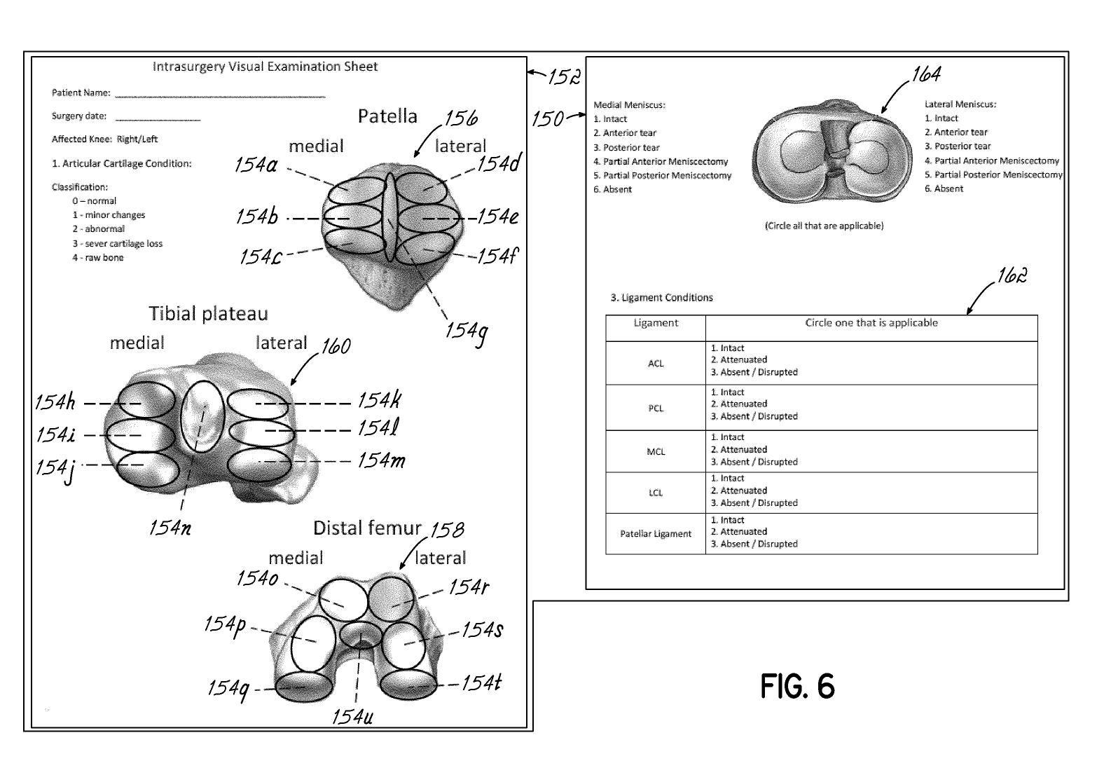

In block 16, the movement of the joint is tracked. This tracking may be via signals received from one or more Inertial Measurement Units (IMUs) attached to the patient, or some other suitable form of tracking, such as with an optical or electromagnetic tracking system. In any case, in block 18, the motion and joint vibration signals are received in the computer, which proceeds to analyze the signals. The detected motion signals may be used to adjust the orientation of the 3-D joint model and to determine the kinematics of the joint. The vibration signals may be used to generate a vibroarthrogram and/or a corresponding acoustic or sound signature that may be listened to or analyzed automatically. The kinematics and vibroarthrogram may then be synchronized to each other and used to analyze the joint. This analysis may include displaying images representing the orientation of the 3-D joint model, vibrations and the accompanying sound generated by the joint as the joint is moved through a range of motion. This analysis may also include grid wise graphical/visual representations of the joint capsule condition recorded with a clinical evaluation sheet on said 3-D joint model that co-relate to the vibration and acoustic analysis pertaining to said joint, as will be described in more detail with respect to FIG. 6. In block 20, the condition of the joint may be determined automatically based on the aforementioned vibration analysis, acoustic analysis, kinematic analysis, or a combination of the three.

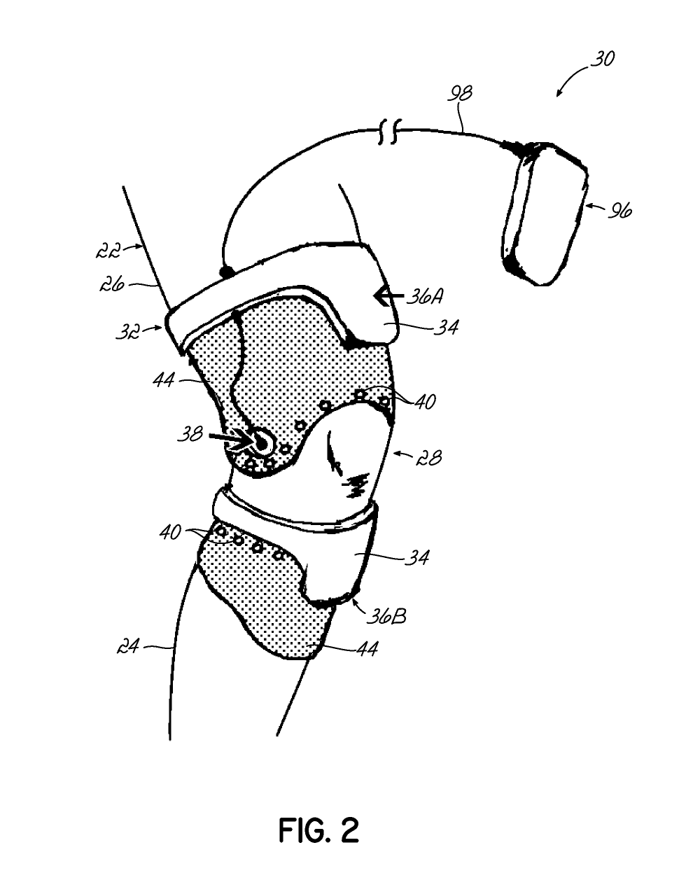

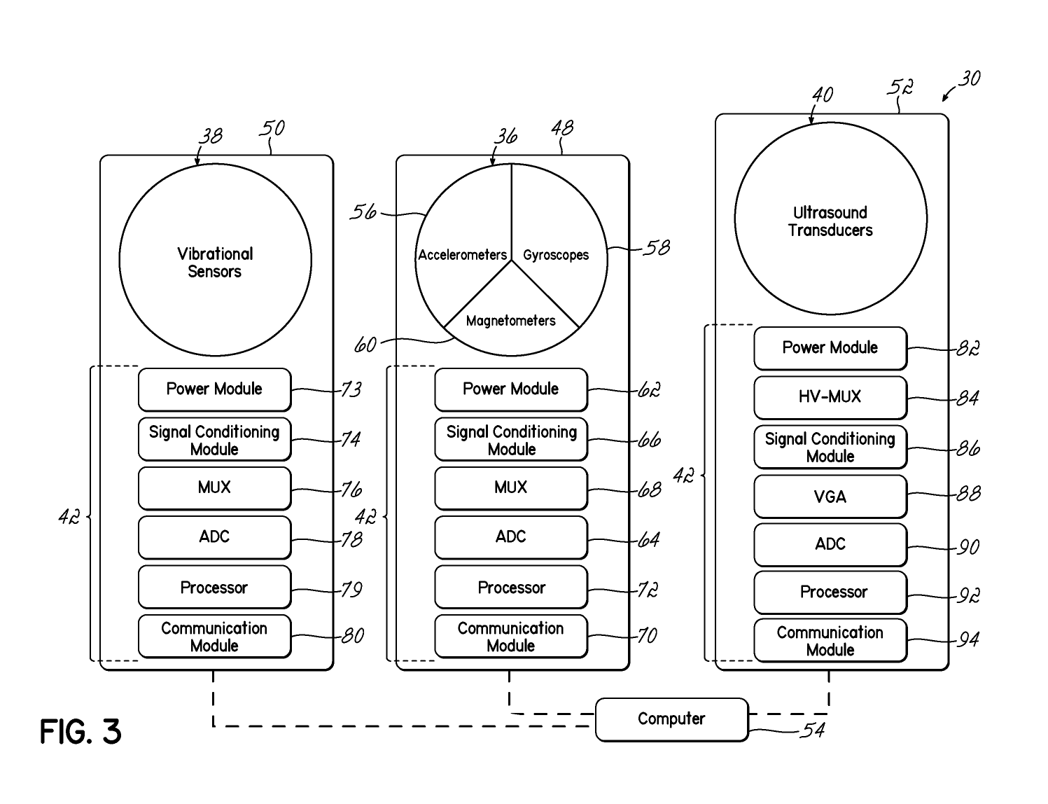

Referring now to FIG. 2, in accordance with an exemplary embodiment of the invention, a patient leg 22 is shown including a shank 24 and thigh 26 joined by a knee joint 28. A joint monitoring apparatus 30 is depicted in the form of a knee brace 32 for use in monitoring and tracking motion of the knee joint 28. The knee brace 32 may include a housing 34 that supports the joint monitoring apparatus 30. The housing 34 may provide a location for one or more inertial measurement unit (“IMU”) sensors 36A, 36B, one or more vibration sensors 38, one or more ultrasound transducers 40, and signal processing circuitry 42 (FIG. 3) related to each of the IMU sensors 36, the vibration sensors 38, and the ultrasound transducers 40. The housing 34 may also include at least one flexible segment 44 configured to secure the knee brace 32 to the leg 22. The flexible segment 44 may include one or more layers of elastic material having an intermediate layer (not shown) that is proximate to the patent's skin and that serves as an acoustic impedance matching layer. The one or more layers of elastic material may thereby facilitate transmission of an ultrasound pulse into the knee joint 28 from the ultrasound transducers 40. The knee brace 32 may also include elastic straps (not shown) with facing materials having hooks and loops (commonly known as VELCRO) for securing the brace 32 to the patient.

Referring now to FIG. 3, a schematic of the joint monitoring apparatus 30 is illustrated showing an inertial monitoring unit 48, a vibration detection module 50, and an ultrasound module 52 operatively coupled to a computer 54. The inertial monitoring unit 48 may detect motion using the inertial monitoring sensors 36. As compared with position tracking systems that rely on optical or electromagnetic localization, the inertial monitoring sensors 36 do not require external observation units. Rather, the inertial monitoring sensors 36 include a plurality of sensors that detect motion unilaterally, thereby allowing the inertial monitoring unit 48 to operate without the need for external reference signals. The inertial monitoring sensors 36 in the exemplary embodiment include, but are not limited to, one or more accelerometers 56, gyroscopes 58, and magnetometers 60.

In an exemplary embodiment of the invention, the inertial monitoring sensors 36 may include an accelerometer 56 that is sensitive to static forces, i.e., an accelerometer configured to output a DC voltage in response to being subjected to a constant acceleration. Thus, the accelerometer 56 may be sensitive to the constant force of gravity. The accelerometer 56 may also include a sensing axis so that the accelerometer 56 generates an output indicating a force of 1 G when the accelerometer sensing axis is perpendicular to the force of gravity. As the accelerometer sensing axis is tilted, the force of gravity acts at an angle to the axis. In response to tilting the sensing axis, the output signal may decrease, indicating a lower sensed level of acceleration. This decrease may continue until the accelerometer sensing axis is positioned parallel to the force of gravity, at which point the signal may reach an output level indicative of a force of 0 G. Accordingly, the relationship between gravity and the accelerometer sensing axis may be used to determine a tilt angle of the accelerometer 56 with respect to the local gravitational field. In an alternative embodiment of the invention, the accelerometer 56 may be a three axis accelerometer having three orthogonal accelerometer sensing axes. In this embodiment, the accelerometer 56 may be configured to monitor the tilt angle for each of the three accelerometer sensing axes relative to the local gravitational field.

The gyroscope 58 may be configured to monitor an angular motion of a gyroscopic sensing axis relative to a local IMU frame. To this end, the gyroscope 58 may generate an output indicative of an angular velocity being experienced by the gyroscopic sensing axis. Thus, a change in the angle of the gyroscopic sensing axis relative to an initial orientation of the inertial monitoring unit 48 may be determined based on the output signal. This change in the angle of the gyroscopic sensing axis may, in turn, be used to determine the angular orientation of the inertial monitoring unit 48 and the orientation of the brace 32 in a known manner. That is, the gyroscope 58 generates an output relative to the angular velocity experienced by the gyroscopic sensing axis. Thus, repositioning the gyroscopic sensing axis relative to an initial orientation may be calculated in accordance with the Newton's equations of angular motion:

∠=∫ωΔt=∠ᵢ+ωΔt

where ∠ is the angle of orientation, and ∠ᵢ is the orientation from previous state.)

The magnetometer 60 may generate one or more output signals indicative of the strength and/or orientation of a magnetic field relative to the magnetometer 60. The magnetometer 60 may thus be configured to serve as a compass and/or magnetic field monitor that detects relative motion between a magnetic sensing axis of the magnetometer 60 and a local magnetic field. The outputs generated by the magnetometer 60 may thereby represent changes in a magnetic field experienced on each magnetic sensing axis. In use, at least two magnetic sensing axes may be used to determine an angle between the inertial monitoring unit 48 and the axis of the magnetic field lines passing through the magnetometer 60. If one of the two magnetic sensing axes becomes insensitive to the local magnetic field (e.g., one of the two magnetic sensing axes is rotated to a position that is orthogonal to the magnetic field), then a third magnetic sensing axis may be used to determine the angle. In an alternative embodiment, tilt angles may be determined from one or more output signals of the accelerometers 56. These tilt angles may in turn be used to compensate for the effects of tilting the magnetic sensing axis.

The inertial monitoring unit 48 may further include a power module 62, an analog-to-digital converter (ADC) 64, a signal conditioning module 66, a multiplexer 68, a communication module 70, and a processor 72. The power module 62 may include circuitry configured to provide power to the components of the inertial monitoring unit 48, e.g., a +3.3 V and/or a +5 V direct current (DC) power source. The power module 62 may also provide a reference voltage to the ADC 64.

The signal conditioning module 66 may couple the output of the inertial monitoring sensors 56 to the ADC 64, and may be configured to reduce noise in the signals provided to the processor 72 from the ADC 64 by amplifying the signals provided to the ADC 64. The level of the signals provided to the ADC 64 may thereby be adjusted so that their amplitudes are within a desired operating range of the ADC 64 input. To this end, the signal conditioning module 66 may provide optimal input signals to the ADC 64 using one or more analog circuits. For example, the signal conditioning module 66 may include a low pass filter, such as a passive low pass filter for reducing high frequency noise from the IMU sensor outputs and to prevent aliasing, and/or an amplifier to amplify the sensor signal to be within a desired input range of the ADC 64.

The multiplexer 68 may include a plurality of inputs, with one input operatively coupled to each of the outputs of the inertial monitoring sensors 36. The multiplexer may also include a single output operatively coupled to an input of the ADC 64. The multiplexer 68 may operate as a high frequency analog switch that sequentially couples the signals at each of the plurality of multiplexer inputs to the multiplexer output. The multiplexer 68 may thereby serialize signals received on multiple inputs into a single time-division multiplexed output that is provided to the input of the ADC 64. In an exemplary embodiment of the invention, the multiplexer 68 may multiplex 16 output signals from the inertial monitoring sensors 36 into one input that is coupled to the ADC 64. The output generated by the multiplexer 68 may be converted into a corresponding digital signal by the ADC 64. In an exemplary embodiment of the invention, the ADC 64 may include a high resolution converter that converts the analog input into a digital signal having a resolution of 24 bits per sample. Alternatively, a lower resolution ADC 64 (e.g., a 16 bit converter) may be used to achieve a higher processing speed and/or a greater sampling rate.

The communication module 70 may include a wireless communication circuit that transmits the digital data generated by the ADC 64 to the computer 54 over a wireless link. The communication module 70 may operate, for example, on one of three frequency bands (e.g., 400 MHz, 916 MHz, and 2.4 GHz) approved by the Federal Communications Commission for unlicensed medical and scientific applications. The communication module 70 may use any suitable wireless or wired communication protocol, such as IEEE 802.15.1 (Bluetooth®), X.25, IEEE 802.11 (WiFI), or a custom protocol such as ultra-wideband (UWB) communication as appropriate depending on the application, to encode the digital data for transmission to the computer 54. Protocols may include signaling, authentication, communication with multiple inertial monitoring units 48, and error detection and correction capabilities.

The processor 72 may be configured to operatively couple and control the ADC 64, the multiplexer 68, and the communication module 70. The processor 72 may acquire digital data from the output of the ADC 64, package the data into data packets, and send the data packets to the communication module 70 for transmission to the computer 54. In an embodiment of the invention, the processor 72 may be a low power processor in order to minimize power consumption of the joint monitoring apparatus 30. In an alternative embodiment, the processor 72 may be a higher powered processor, such as a digital signal processor (DSP) or application specific integrated circuit (ASIC) so that the processor 72 may be used to perform digital signal processing, such as data compression, prior to transmitting the data to the computer 54. Multiple core or multiple processor architectures, and or a field programmable gate array (FPGA) may also be used.

Similarly as described above with respect to the inertial monitoring unit 48, the vibration detection module 50 may include one or more vibration sensors 38, and signal processing circuitry 42 comprising a power module 73, a signal conditioning module 74, which may include a charge amplifier (not shown), a multiplexer 76, an ADC 78, a processor 79, and a communication module 80. The signal processing circuitry 42 of vibration detection module 50 may operate to provide signals generated by the vibration sensors 38 to the computer 54. The vibrations detected by the vibration sensors 38 may thereby be used to provide insight into the condition of a patient's joint, such as the knee joint 28 in FIG. 2. To this end, the one or more vibration sensors 38 may be used to collect vibrations generated by the joint. These vibrations may be used to generate a vibration signature (i.e., a pattern or plot of vibration amplitude verses time, or vibroarthrograph) and an acoustic signature (i.e., an audio signal or pattern that may be listened to or analyzed via signal processing). These vibration and acoustic signatures may characterize femur and tibia interaction (or other bones forming a joint, as the case may be) during patient activities. The vibration and acoustic signatures generated during knee motion may thereby be used to help differentiate a healthy patient from an osteoarthritic patient. The vibration and acoustic signatures may also be used to determine various soft tissue defects in the joint, such as meniscul and ligament injuries, patellar clunk/crepitus/chondromalacia etc. in the knee joint and the condition of the labrum, and/or injuries to the hip joint ligaments in the hip joint. The vibration and acoustic signatures may further be used to determine abnormal conditions in artificial implants, such as severe cam-post impact, condylar lift-off, and unexpected wear patterns in the knee joint or total hip arthroplasty squeaking and metal particle incursion in the hip joint. The observed vibration and its accompanying sound may thus provide a useful indicator for diagnosing the condition of the joint.

One exemplary vibration sensor 38 is a dynamic accelerometer, which is a type of accelerometer configured to detect rapid changes in acceleration, such as may be associated with vibrations generated by a moving joint. In an embodiment of the invention, the joint monitoring apparatus 30 may be configured so that the vibration sensor 38 is detachable to allow positioning of the sensor 38 in proximity to a desired portion of the joint. As best shown in FIG. 1, the detachable vibration sensor 38 may be placed near the knee joint 28 and secured with adhesives to monitor vibration while the knee joint 28 is in motion. As compared to static accelerometers, dynamic accelerometers are not necessarily sensitive to a static accelerative force, such as gravity. However, in an alternative embodiment of the invention, accelerometers having a wide frequency range may be used to detect both patient motion and joint vibration, so that both these signals are provided to the computer 54 from a single accelerometer. The power module 73 may include, for example, a +3.3 V DC source, a +5 V DC source, and power source having an output voltage between +18 V and +30 V (e.g., +24 V) DC. The power module may also provide a precision voltage reference to the ADC 78

The signal generated by the vibration sensors 38 may be processed by the signal conditioning module 74 before entering the multiplexer 76 and ADC 78, similarly as described above with reference to the inertial monitoring sensors 36. As compared to the ADC 64 of the inertial monitoring unit 48, the ADC 78 of vibration detection module 50 may have a higher sample rate to capture the higher frequency signals generated by the vibration sensors 38 (e.g., a sample rate above the Nyquist rate for the desired bandwidth of the vibration sensor output signals). To this end, the ADC 78 of vibration detection module 50 may be selected to trade resolution for a higher sample rate. The digital data output by the ADC 78 may be coupled to the communication module 80 for processing and transmission to the computer 54 similarly as described above with respect to the inertial monitoring unit 48.

The processor 79 may be configured to control the components of the vibration detection module 50, as well as receive the digitized output signal from the ADC 78, package the received data into data packets, and send the data packets to the communication module 80 for transmission to the computer 54. Similarly as discussed with respect to inertial monitoring unit 48, the processor 79 may be any suitable processor, such as a low power processor in order to minimize power consumption of the joint monitoring apparatus 30. In an alternative embodiment, the processor 79 may be a higher powered processor, such as a digital signal processor (DSP) or application specific integrated circuit (ASIC) so that the processor 72 may be used to perform digital signal processing, such as data compression, prior to transmitting the data to the computer 54. Multiple core or multiple processor architectures, and or a field programmable gate array (FPGA) may also be used.

The communication module 80 may include a wireless communication circuit that transmits the digital data generated by the ADC 78 to the computer 54 over a wireless link. The communication module 80 may operate, for example, on one of three frequency bands (e.g., 400 MHz, 916 MHz, and 2.4 GHz) approved by the Federal Communications Commission for unlicensed medical and scientific applications. The communication module 80 may use any suitable wireless or wired communication protocol, such as Bluetooth®, X.25, WiFI, or a custom protocol such UWB communication as appropriate depending on the application, to encode the digital data for transmission to the computer 54. Protocols may include signaling, authentication, communication with multiple vibration detection modules, and error detection and correction capabilities.

The ultrasound module 52 may include one or more ultrasound transducers 40, a power module 82, a high voltage multiplexer 84, a signal conditioning module 86, a multi-channel variable gain amplifier (VGA) 88, an ADC 90, a processor 92, and a communication module 94. The ultrasound transducers 40 may include a plurality of pulse echo mode ultrasound transducers arranged in the flexible segment 44 of knee brace 32. Each ultrasound transducer 40 may be comprised of a piezoelectric crystal configured to emit an ultrasound pulse in response to an electrical signal. The ultrasound pulse may be transmitted from the ultrasound transducer 40 through the skin and soft tissues of the patient. When the ultrasound pulse reaches a boundary between tissues having different acoustic impedance properties, such as an interface between bone and a soft-tissue, an echo is generated and reflected back to the ultrasound transducer 40. The time delay between an initial echo (i.e., the echo generated by the interface between the flexible segment 44 of knee brace 32 and the skin) and an echo generated by the bone-tissue interface may be used to determine a distance between the ultrasound transducer 40 and the bone. By including one or more ultrasound transducers 40 in the brace 32, the relative motions between the knee brace 32 and the patient's bones may be determined as is described in greater detail in U.S. Application Pub. No. 2012/0029345, filed on Aug. 2, 2011 and entitled “NONINVASIVE DIAGNOSTIC SYSTEM”, the disclosure of which is incorporated herein by reference in its entirety.

In addition to providing power to the ultrasound module 52, the power module 82 may include a high voltage pulse generator configured to excite the ultrasound transducers 40 with ultrasound bursts via the high voltage multiplexer 84. To this end, the high voltage multiplexer 84 may include an analog switch configured to selectively couple the high voltage output of the high voltage pulse generator to one or more of the plurality of ultrasound transducers 40.

The signal conditioning module 86 may be coupled to (or include) the multi-channel VGA 88, which may provide a time-based variable gain control over the received echo signals generated by the ultrasound transducers. Normally the transmitted ultrasound pulse and the returning echo are attenuated by soft tissue as each signal propagates through the human body. Accordingly, after the ultrasound transducer 40 emits the ultrasound pulse, the amplitude of the pulse is attenuated as the signal passes through the patient. Thus, echo signals originating from deep within the patient tend to have lower amplitude than those originating from close to the surface due to their longer propagation path. A received echo signal that initially has sufficient amplitude to be encoded by the ADC 90 may therefore fade into the background noise by the end of the ultrasound scanning or receiving period. To address this issue, the VGA 88 may be configured to dynamically increase the gain applied to the received echo signal over the receiving period to compensate for this varying attenuation. The gain may also be varied across the inputs to the VGA 88 so that the gain may be adjusted independently for each ultrasound transducer 40 coupled to the VGA 88. The VGA 88 may thereby improve the reliability and quality of the echo signal conversion by the ADC 90 as compared to systems lacking this dynamic gain feature.

The ADC 90 of ultrasound module 52 may be similar to the ADCs 64, 78 of inertial monitoring unit 48 and vibration detection module 50. However, because the ADC 90 is responsible for converting the echo signal of an ultrasound pulse into to a digital signal, the ADC 90 may require a higher sampling frequency than either the ADC 64 of inertial monitoring unit 48 or the ADC 78 of vibration detection module 50. This higher conversion rate may be required because the bandwidth of the ultrasound pulse is significantly higher than signals generated by either the inertial monitoring sensors 36 or vibration sensors 38. In any case, the output signal generated by the ADC 90 may include an array of digital data points, or samples representing the analog echo signal similarly as described above with respect to the other ADCs 64, 78.

The processor 92 may be configured to control the components of the ultrasound module 52, as well as receive the digitized output signal from the ADC 90, package the received data into data packets, and send the data packets to the communication module 94 for transmission to the computer 54. Similarly as discussed with respect to inertial monitoring unit 48, the processor 92 of ultrasound module 52 may be any suitable processor. In an embodiment of the invention, the processor 92 may be a DSP, ASIC, multiple core processor, and/or may include multiple processors configured to process the digital signal generated from the ultrasound transducers 40 into physical units indicative of the distance between the ultrasound transducer 40 and the bone surface. Signal processing may thereby be performed in the ultrasound module 52 prior to transmission of the processed data to the computer 54. This processing may reduce the amount of data that must be transmitted to, and the processing load on, the computer 54. In any case, and similarly as described above with respect to the inertial monitoring unit 48, the communication module 94 may include a wireless communication circuit that transmits the digital data generated by the ADC 90 and/or processor 92 to the computer 54 over a wireless link.

The modules 48, 50, 52 may receive power from batteries incorporated into the housing of the knee brace 32. In an alternative embodiment, the modules 48, 50, 52 may receive power from an external power source 96 coupled to the brace 32 via a power line 98. Using an external power source 96 may reduce the size and weight of the joint monitoring apparatus 30 as well as allow the use of higher performance circuitry.

One or more of the communication modules 70, 80, 94 may be incorporated into the housing of the knee brace 32. In an alternative embodiment, to reduce the size and weight of the joint monitoring apparatus 30, one or more of the communication modules 70, 80, 94 may also be external to the knee brace 32, and may communicate with the electronic components of the modules 48, 50, 52 wirelessly or via one or more wires tethering the one or more communication modules 70, 80, 94 to the knee brace 32. In embodiments having external communication modules, the communication modules may be integrated into the external power source 96. In embodiments including communications modules 70, 80, 94 employing wireless communication links, the communications modules 70, 80, 94 may operate, for example, on one of three different bandwidths (e.g., 400 MHz, 916 MHz, and 2.4 GHz) that are approved by the Federal Communications Commission for medical and scientific applications. As discussed with respect to the communication module 70 of inertial monitoring unit 48, the communication modules 70, 80, 94 may use any suitable wireless or wired communication protocol, such as IEEE 802.15.1 (Bluetooth®), X.25, IEEE 802.11 (WiFI), or a proprietary protocol as appropriate depending on the application, to encode the digital data for transmission to the computer 54. Protocols may include signaling, authentication, communication with multiple inertial monitoring units 48, and error detection and correction capabilities.

In an embodiment of the invention, the accelerometer 56, the gyroscope 58, and the magnetometer 60 may be separated into distinct sensor circuit layouts to increase the modularity and customizability of the inertial monitoring unit 48. Furthermore, the sensitivities of the inertial monitoring sensors 36 in the inertial monitoring unit 48 may be designed to perform within a finite sensitivity range and boundary conditions. For example, the gyroscope 58 may have a sensitivity rating selected to accommodate an expected maximum measurable angular motion for a particular application. Because each motion performed by the joint under study has a different kinematic characteristic (for example, the shank 24 has far less motion during a rising motion from a chair as compared with walking), selecting the components of the inertial monitoring unit 48 in accordance with a selected capability for a particular motion may optimize the performance of the joint monitoring apparatus 30.

Moreover, segmenting the circuit layouts allows for greater adaptability of the inertial monitoring unit 48 for use in analyzing the motion of another portion of the patient's body. That is, while the illustrative embodiment is specifically drawn to the knee joint, other joints (such as the hip, the shoulder, and the spine) may exhibit significantly different kinematics as compared with the knee. The modular design of the inertial monitoring unit 48 and the inertial monitoring sensors 36 provides for a quick and easy adjustment of the motion tracking apparatus 30 by enabling the switching or exchange of one component for another having a different selected sensitivity range that is better suited for evaluating the joint or movement in question.

Additionally, while the illustrative embodiment of the present invention is specifically described as including one accelerometer 56, one gyroscope 58, and one magnetometer 60, those having ordinary skill in the art will understand that the inertial monitoring unit 48 may have other combinations and numbers of inertial monitoring sensors 36. Thus, inertial monitoring units 48 in accordance various embodiments of the present invention may include any combination of components, including, for example, two accelerometers 56, two gyroscopes 58 each with a different operational dynamic range, and one magnetometer 60. The selection of components may be based, in part, on the particular need or preference of the evaluating physician, the joint to be evaluated, the range of motion of the patient, the expected rate of motion (slow versus fast movement or rotation), and/or the range of motion permitted in the evaluation setting (examination room versus surgical table). Apparatuses, systems and methods for monitoring a joint are also described in concurrently filed U.S. patent application entitled “MOTION TRACKING SYSTEM WITH INERTIAL-BASED SENSING UNITS”, Attorney Docket No. JVUE-6CIP1, the disclosure of which is incorporated herein by reference in its entirety.

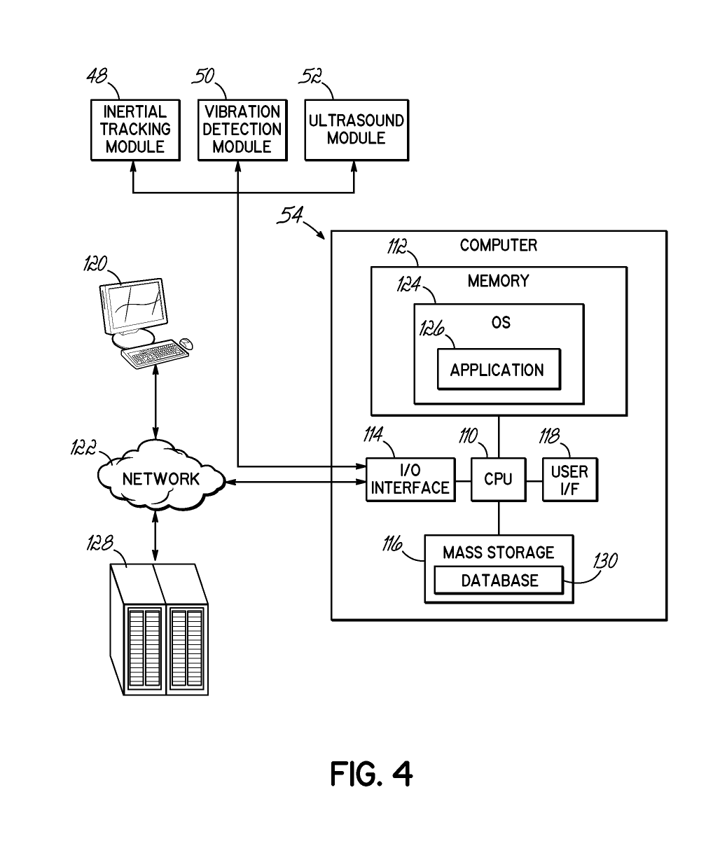

Referring now to FIG. 4, the computer 54 may include a processor 110, memory 112, an input/output (I/O) interface 114, a mass storage device 116, and a user interface 118. The computer 54 may be considered to represent any suitable type of computer, computing system, server, disk array, or programmable devices such as a handheld device, a networked device, or an embedded device, etc. The computer 54 may be in communication with one or more networked computers 120 via one or more networks 122, such as a cluster or other distributed computing system, through the I/O interface 114.

The processor 110 may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in the memory 112. Memory 112 may be a single memory device or a plurality of memory devices including but not limited to read-only memory (ROM), random access memory (RAM), volatile memory, non-volatile memory, static random access memory (SRAM), dynamic random access memory (DRAM), flash memory, or cache memory. Memory 112 may also include a mass storage device such as a hard drive, optical drive, tape drive, non-volatile solid state device, or any other device capable of storing digital information.

The processor 110 may operate under the control of an operating system 124 that resides in memory 112. The operating system 124 may manage computer resources so that computer program code embodied as one or more computer software applications, such as an application 126 residing in memory 112 may have instructions executed by the processor 110. In an alternative embodiment, the processor 110 may execute the applications 126 directly, in which case the operating system 124 may be omitted.

The mass storage device 116 typically includes at least one hard disk drive and may be located externally to the computer 54, such as in a separate enclosure or in one or more networked computers 120, one or more networked storage devices 128 (including, for example, a tape or optical drive), and/or one or more other networked devices (including, for example, a server). The mass storage device 116 may also host one or more databases 130.

The user interface 118 may be operatively coupled to the processor 110 of computer 54 in a known manner to allow a system operator to interact directly with the computer 54. The user interface 118 may include output devices such as video and/or alphanumeric displays, a touch screen, a speaker, and any other suitable audio and visual indicators capable of providing information to the system operator. The user interface 118 may also include input devices and controls such as an alphanumeric keyboard, a pointing device, keypads, pushbuttons, control knobs, microphones, etc., capable of accepting commands or input from the operator and transmitting the entered input to the processor 110.

Those skilled in the art will recognize that the computing environment illustrated in FIG. 4 is not intended to limit the present invention. In addition, various program code described herein may be identified based upon the application or software component within which it is implemented in a specific embodiment of the invention. However, it should be appreciated that any particular program nomenclature that follows is used merely for convenience, and thus the invention should not be limited to use solely in any specific application identified and/or implied by such nomenclature. It should be further appreciated that the various features, applications, and devices disclosed herein may also be used alone or in any combination. Moreover, given the typically endless number of ways in which computer programs may be organized into routines, procedures, methods, modules, objects, and the like, as well as the various ways in which program functionality may be allocated among various software layers that are resident within a typical computing system (e.g., operating systems, libraries, APIs, applications, applets, etc.), and/or across one or more hardware platforms, it should be appreciated that the invention is not limited to the specific organization and allocation of program or hardware functionality described herein.

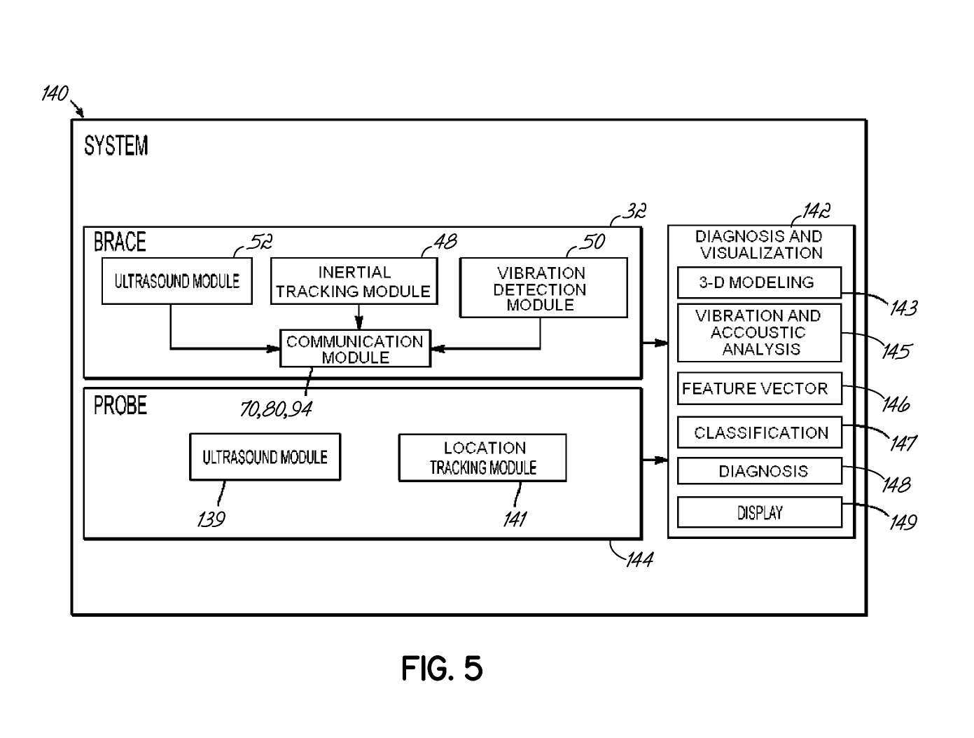

Referring now to FIG. 5, an exemplary diagnostic system 140 is presented in accordance with an embodiment of the invention. The diagnostic system 140 may include the brace 32, a diagnosis and data visualization module 142, and an ultrasound probe 144 for imaging the joint 28 during diagnosis (if necessary) and to register the bones in the joint 28 to the 3-D model in computer 54. The inertial tracking module 48, ultrasound module 52, and ultrasound probe 144 may collectively provide joint kinematics tracking information to the diagnosis and data visualization module 142. To this end, the ultrasound probe 144 may include an ultrasound module 139 that obtains ultrasound data from the patient, and a location tracking module 141 that provides probe location data to the diagnosis and data visualization module 142. The diagnosis and data visualization module 142 may in turn manipulate the patient-specific 3-D model based on the received data to provide kinematic data to the system user via a display module 149. The diagnosis and data visualization module 142 may also receive vibration data from the vibration detection module 50 while the joint 28 is in motion.

The diagnosis and visualization module 142 may include program code executed on the computer 54 in the form of one or more applications 126, databases 130, and/or modules. These applications, databases, and modules may include a 3-D modeling module 143, a vibration and acoustic analysis module 145, a feature vector module 146, a classification module 147, a diagnosis module 148, and the aforementioned display module 149. The 3-D modeling module 143 may build and access an atlas or database of 3-D joint models, and may receive and process joint position data from the brace 32 to synchronize a selected 3-D joint model with the position of the actual patient joint. The 3-D modeling module 143 may also generate kinematic data for use by the diagnosis module 148.

The vibration and acoustic analysis and feature vector modules 145, 146 receive and analyze vibration data from the brace 32. These modules may also provide data to the classification module 147 regarding statistical features of a vibroarthrogram and the acoustic signature generated from the vibration data. The classification module 147 may, in turn, determine a level of correlation or correspondence between the statistical features of the generated vibroarthrogram acoustic signature, and statistical features of vibroarthrograms acoustic signatures indicative of known joint conditions, which may be stored in a database of vibroarthrograms acoustic signatures. The classification module 147 may also determine scores for each of a plurality of feature vectors based on the statistical features of the vibroarthrograms.

The diagnosis module 148 is configured to identify a condition of the knee joint 28 based on the output of the classification module 147. The diagnosis module 148 may also mathematically describe the relative motion of the bones in the knee joint 28 as such motion is tracked by the 3-D modeling module 143. The kinematics of the knee may then be correlated with a database of mathematical descriptions of joint motion that includes descriptions of healthy and clinically undesirable joint motion to identify possible conditions in the knee 28.

During a dynamic activity, the interaction between the moving articulating surfaces of the joint may induce vibrations of the bones. In a healthy joint, the articulating surfaces are smooth and the vibration is minimal. But as the cartilage degenerates or there is injury to other soft tissue structures, vibrations increase, and may become audible. Loss of cartilage is a natural process of aging but may not necessarily be severe enough to cause pain. However, if the cartilage deteriorates due to arthritis, its loss is accelerated and most often causes unbearable pain that limits the mobility of afflicted patients. In case of a complete loss of articular cartilage, the raw bone surfaces interact with each other directly so that the joint produces an identifiable vibration and/or acoustic signatures. In the case of soft tissue damage, the injury may result in the change of the vibration and acoustic signatures in a unique injury specific manner. This unique injury specific change may then be detected through the changes in the vibroarthogram and/or the acoustics produced by the vibration signals when compared to other healthy patterns.

To create a vibroartrography database 130 of joint vibrations, vibration data may be collected from a first plurality of test subjects having healthy knees and a second plurality of test subjects suffering from a joint condition, such as knee arthritis or other soft tissue and/or ligament injuries. To further augment the vibroartrography database, during a subsequent joint replacement procedure or investigative arthroscopic procedure on test subjects selected from the second plurality of patients, the surgeon may examine the condition of the articular cartilage and soft tissue structures of the joint and record an assessment of the joint condition. To this end, the surgeon's observations may be recorded on an intrasurgical evaluation sheet during a joint procedure to provide the information about the location and severity of the articular cartilage damage. Other factors that might affect the vibrations generated by the joint, such as ligament deficiency or meniscus injuries may also be examined and described.

Referring now to FIG. 6, in an exemplary embodiment of the invention, an intrasugical evaluation sheet 150 for a knee joint may include a section 152 for recording articular cartilage condition that includes five numerical classifications. These classifications may be assessed for each of a plurality of regions 154a-154u of the medial and lateral patella 156, distal femur 158, and tibial plateau 160. The classifications may include, for example: (0) normal; (1) minor changes; (2) abnormal; (3) severe cartilage loss; and (4) raw bone. The evaluation sheet 150 may also include a section 162 for recording ligament conditions for the Anterior Cruciate Ligament (ACL), Posterior Cruciate Ligament (PCL), Medial Collateral Ligament (MCL), Lateral Collateral Ligament (LCL) and patellar ligament as being either: (1) intact; (2) attenuated; or (3) absent/disrupted. The evaluation sheet may further include a section 164 for recording the condition of the medial meniscus and lateral meniscus as one of: (1) intact; (2) having an anterior tear; (3) having a posterior tear; (4) having been subject to a partial anterior meniscectomy; (5) having been subject to a partial posterior meniscectomy; or (6) absent.

The vibroarthrography database 130 may be augmented by associating the surgical observations entered on the evaluation sheet 150 with the corresponding recorded joint vibration and acoustic signatures for the joint in question. This information may then be converted to grid based coded graphic/visual display and provide input to the display module 149 of the diagnostic system 140. Embodiments of the invention may thereby provide insight into the exact condition of the cartilage and the amount of damage at every compartment of the joint, as well as any other factors passably altering the vibration pattern, such as any ligament deficiency or meniscal injuries. The visual display may further provide information on the exact interacting locations (and their condition as recorded on the evaluation sheet of FIG. 6) of the femur/tibia/patella that interact to produce a specific vibroathrographic pattern. Having this information may facilitate correlating the condition of the joint with the detected vibration data, and the vibration and acoustic signatures generated there from. Thus, vibroarthrography as used herein may provide an additional source of data that can be collected non-invasively under in-vivo conditions, to enhance the diagnostic capabilities of the diagnostic system 140.

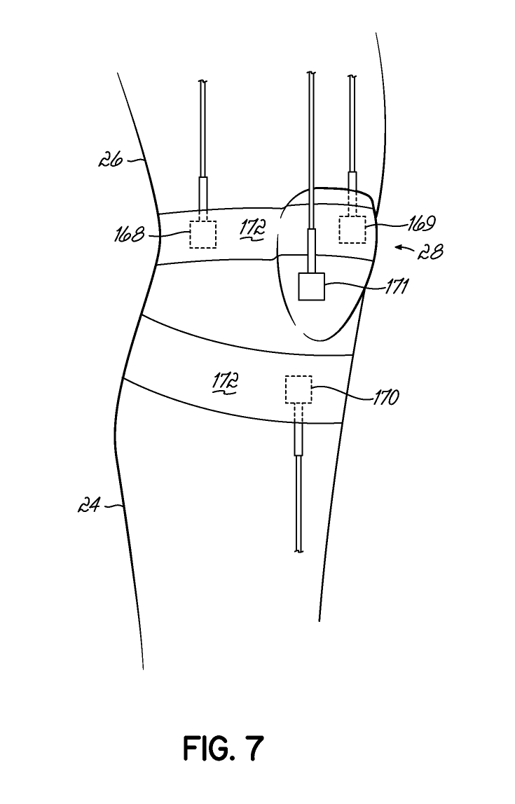

Referring now to FIG. 7, and in accordance with an embodiment of the invention, vibration data may be collected using a plurality (e.g., four) tri-axial accelerometers 168-171 each having a sensitivity of about 100 mV/g, a measurement range of about ±50 g, and a frequency response of about 0.5 to 5 kHz. One such accelerometer suitable for collecting vibration data is a model 356A12 accelerometer available from PCB Piezotronics Inc. of Depew, N.Y. The accelerometers 168-171 may be included in an embodiment of the knee brace 32, or may be attached to the surface of the skin at the lateral femoral epicondyle (accelerometer 168), medial femoral epicondyle (accelerometer 169), the tibial tuberocity (accelerometer 170), and the patella (accelerometer 171) respectively using any suitable means, such as elastic wrap and/or hypoallergenic adhesive tape 172.

As previously described, the accelerometers are coupled to the signal conditioning module 74, which may have a gain of about 10 dB and include a low-pass filter with a cut-off frequency of about 4700 Hz. One suitable device for providing the signal conditioning module 74 is a Model 482C signal conditioner available from PCB Piezotronics Inc., Depew, N.Y. The conditioned signal is then coupled to the ADC 78. The ADC 78 may be a multi-channel analog-to-digital converter having a 14 bit resolution and 150-200 kHz waveform recording capability. One suitable device for providing ADC 78 is a Model DI-720, available from DATAQ Instruments Inc. of Akron, Ohio. As the joint 28 is moved to produce vibrations, the movement may be tracked using the inertial tracking module 48 so that the vibration signal data can be synchronized to the joint position as the vibration signal data is generated and stored in memory 112.

In an embodiment of the invention, the accelerometers 168-171 may have sufficient bandwidth so that the signals generated reflect acceleration resulting from both movement of the joint 28 as well as from vibrations of the bones caused by the movement. The accelerometers 168-171 may thereby provide signals for use by both the inertial tracking module 48 and vibration detection module 50. To this end, the raw accelerometer signals may be separated into motion and vibration components. This separation may be achieved by passing the output of the accelerometers 168-171 through one or more low-pass and/or high-pass filters. One suitable filter may be an Infinite Impulse Response (IIR) Butterworth filter configured to have a signal attenuation of 80 dB at a cut-off frequency of 20 Hz. The raw accelerometer signals may be separated by such a filter into a low-frequency band containing motion information, and a high-frequency band containing vibration information.

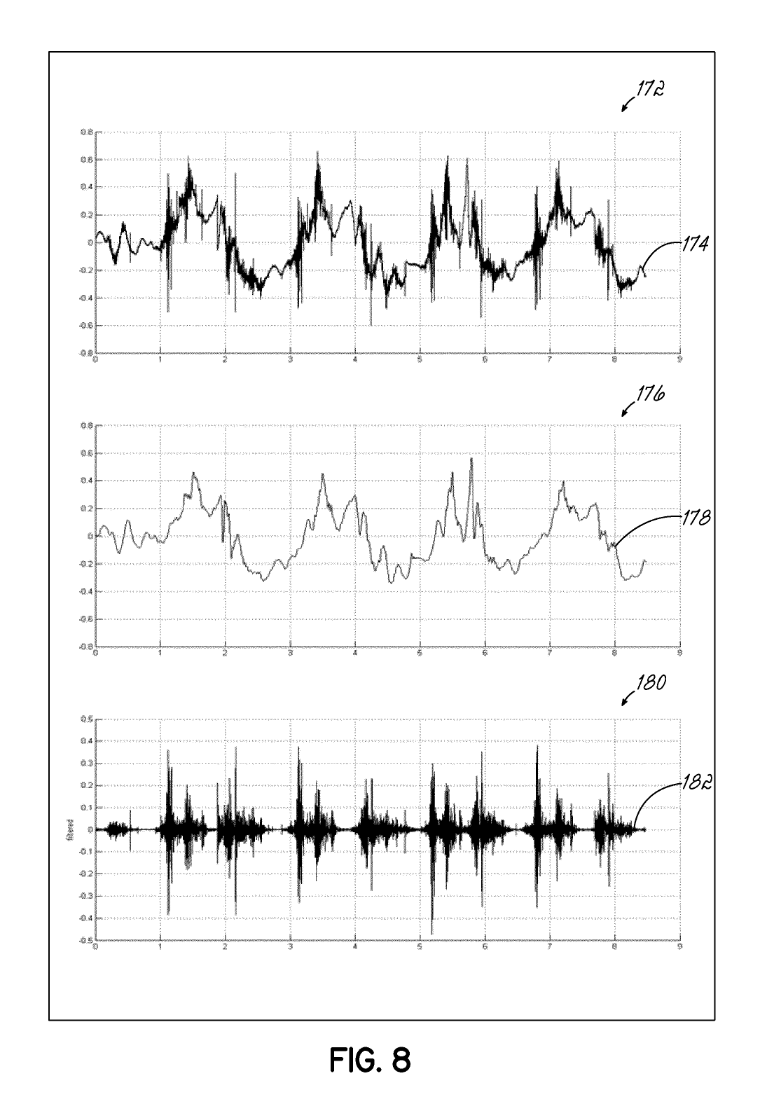

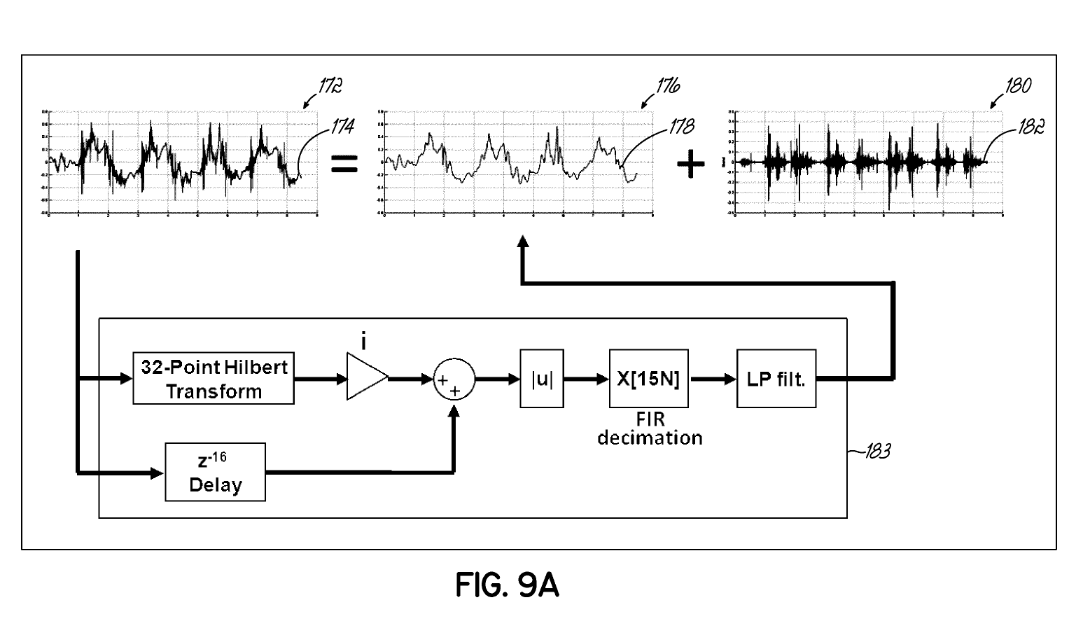

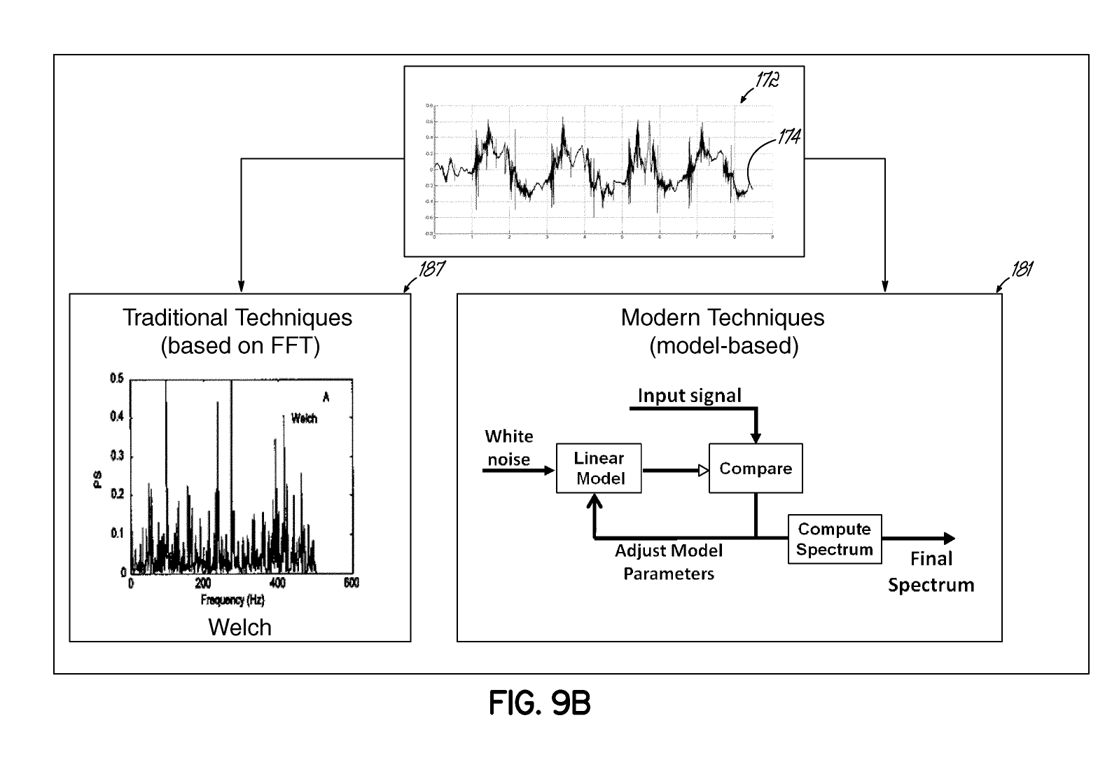

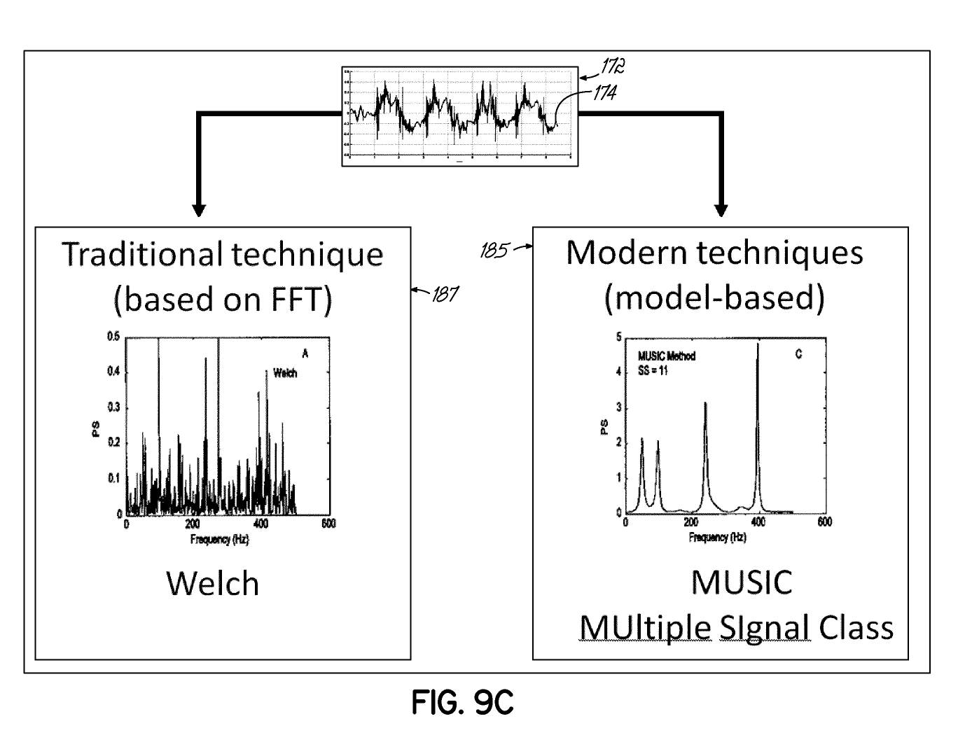

Referring now to FIGS. 8 and 9A-9C, a series of graphs are presented each illustrating a plot of an exemplary signal, or a portion thereof, received from one of the accelerometers 168-171. Graph 172 includes a plot 174 representing a raw signal received from the accelerometer via the signal conditioner. This signal may have been filtered through a low pass filter having, for example, a cut-off frequency of about 4700-5000 Hz to reduce noise and prevent aliasing. Graph 176 includes a plot 178 representing a low-pass filtered portion of the raw signal of plot 174 that includes a motion component of the raw vibration signal. Graph 180 includes a plot 182 representing a high pass filtered portion of the raw signal of plot 174 that includes the vibration portion of the raw signal of plot 174. As can be seen from the plots 174, 176, 178, a low pass filter, such as the filter 183 shown in FIG. 9A, or some other type of signal processing such as shown in FIGS. 9B and 9C, may be used to separate the low frequency portion of the accelerometer output signal from the high frequency portion of the signal.

The low pass filter 183 removes low frequency motion components from the raw signal, thereby yielding a vibroarthrogram suitable for further analysis by the diagnosis and visualization module 142. The resulting vibroarthrogram may also be also converted into audible form and correlated with the motion of the 3-D model for display to the user. Persons having ordinary skill in the art will understand that other signal analysis techniques may also be used to separate the portions of the accelerometer output signals containing motion information from those portions containing vibration information. These techniques may include analysis such as: (1) model based analysis techniques that compute the vibroarthrogram spectrum by comparing the input signal to filtered white noise 181; (2) multiple signal classification (e.g., the MUSIC algorithm) 185 which estimates the frequency content of a signal using an eigenspace method; or (3) traditional spectral analysis, such as with fast Fourier transforms 187 as is known in the art of signal processing.

To reduce the subjectivity of diagnoses based on simply listening to the noise emitted by a moving joint, the diagnosis module 148 uses numerical methods to identify possible injuries. These methods may identify joint condition based on the digital signature of the vibration, and may use pattern recognition techniques. Methods may include time domain analysis or frequency domain analysis. However, the detected vibration signal pattern can be affected by a number of factors including but not limited to: (1) the severity of the joint degeneration; (2) the thickness of the subcutaneous tissue present between the articulating bone and the accelerometer due to the damping effect of the soft tissue; (3) the location of the accelerometer relative to the underlying bones; (4) the type of activity requiring joint motion (e.g., load bearing or free movement); (5) the speed of the activity (faster movements result in higher accelerations); and (6) the direction in which the acceleration is being measured.

Some of these parameters may be controlled to at least some extent (e.g. the speed of the activity), and others may be optimized (direction and location of the accelerometer placement). However, the anatomical diversity and various stages of arthritis may cause significant variations in observed vibration patterns. These variations, in turn, must be accounted for by the diagnosis and visualization module 142 in order to provide accurate diagnosis of joint conditions.

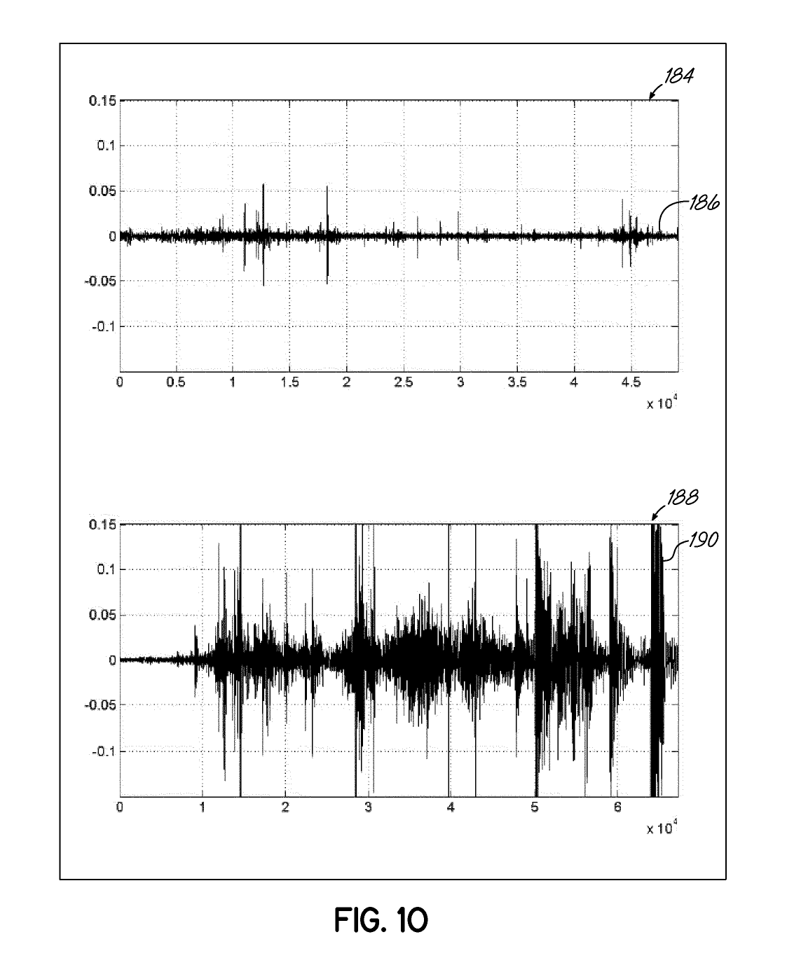

Referring now to FIG. 10, a graph 184 illustrates an exemplary plot 186 of a vibroarthrogram obtained for a healthy subject, and a graph 188 illustrates an exemplary plot 190 of a vibroarthrogram obtained from a subject diagnosed with patellofemoral joint arthritis. As can be seen from the plots 188, 190, the magnitude of the vibration caused by joint movement tends to be higher for a degenerated knee as compared to a healthy knee. Analysis of the plots, or vibroarthrograms 186, 190 may include calculating statistical parameters of the original and rectified vibroarthrograms. These statistical parameters may include, but are not limited to, mean, variance, standard deviation, skewness, kurtosis, signal envelope integral, signal envelope integral as a function of duration, as well as 90th, 95, 97th and 99th quantiles. These statistical parameters may in turn be used to examine features that could be included in a feature vector of the signals. A desirable feature for a statistical parameter is that the statistical parameter provides separation between vibroarthrograms from healthy joints and vibroarthrograms from injured joints. Statistical parameters that have this feature may provide the highest success rate for classifying joint conditions. Although embodiments of the invention are generally described herein with respect to a few statistical parameters, those skilled in the art will recognize that the methods and systems described may also be used with the analysis of other statistical parameters (e.g., entropy, complexity) without departing from the scope of embodiments of the invention.

By way of example, it has been determined that the mean and standard deviations of rectified vibroarthrograms are higher for arthritic than for healthy subjects. Another statistical parameter that may provide separation between vibroarthrograms of injured and healthy joints is the 99th quantile. Once the statistical parameters that provide separation between vibroarthrograms produced by various joint conditions are identified, the parameters may be used to define a feature vector. The feature vector may classify vibroarthrograms by combining the separations provided by multiple statistical parameters into a composite separation, thereby providing improved diagnosis as compared to relying on a single statistical parameter.

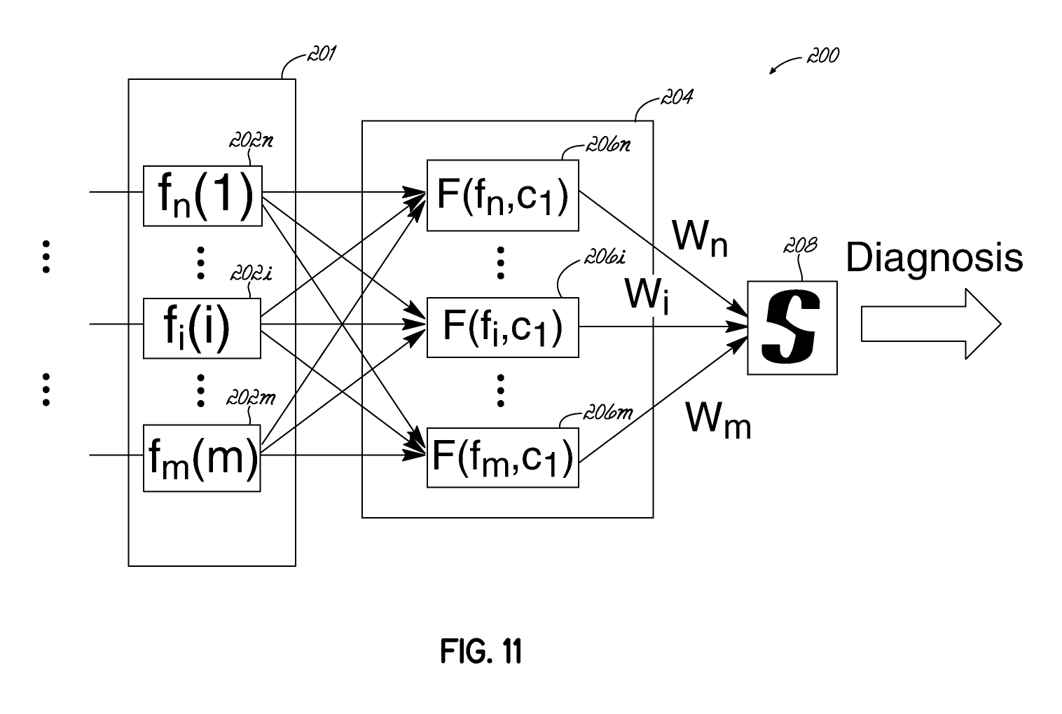

Referring now to FIG. 11, a pattern classifier 200 in accordance with an embodiment of the invention includes a set of statistical parameters that are determined by a signal features module 201. The signal features module 201 may include functions 202n-202m that calculate one or more signal features such as the mean (μ), standard deviation (σ), complexity (FF), skewness (S), kurtosis (K), and/or entropy (H) of a vibroarthrogram which may be calculated using the equations below:

$$\begin{matrix}μ = \sum xP ( x ) & ( 1 ) \\ σ = \sqrt{\frac{1}{N} {\sum }_{i = 1}^{N} {( xi - μ )}^{2}} & ( 2 ) \\ FF = \frac{{M}_{{x}^{′}}}{{M}_{x}} = \frac{{σ}_{{x}^{″}} / {σ}_{{x}^{′}}}{{σ}_{{x}^{′}} / {σ}_{x}} & ( 3 ) \\ S = \frac{{m}_{3}}{{( {m}_{2} )}^{3 / 2}} & ( 4 ) \\ K = \frac{{m}_{4}}{{( {m}_{2} )}^{2}} & ( 5 ) \\ H = - {\sum }_{l = 0}^{L - 1} {p}_{x} ( {x}_{l} ) {log}_{2} [ {p}_{x} ( {x}_{l} ) ] & ( 6 )\end{matrix}$$

The outputs of the statistical parameter functions 202n-202m may be provided to a radial basis function network 204 that includes a plurality of feature vectors 206n-206m. The feature vectors 206n-206m may in turn selectively weigh and combine selected outputs of the statistical parameter functions 202n-202m to calculate a relative value or likelihood that the vibroarthrogram being analyzed corresponds to a joint having a particular condition. That is, each feature vector may include one or more weighted signal features of the vibroarthrogram, and based on these weighted signal features, produce a score that may be compared to scores of other feature vectors. Each feature vector may thereby provide a score indicative of a level of correspondence between the statistical features of the vibroarthrogram and a condition of the joint. The scores of the feature vectors 206n-206m are provided to a selector 208 that determines a diagnosis based on the scores.

In an exemplary embodiment of the invention, based on the selected signal features included in the feature vector, the pattern classifier 200 classifies the pattern of the vibroarthrographic signal to the defective condition of the joint in question To this end, the pattern classifier 200 may use a minimum-error-rate classification to identify the group to which the vibroarthrographic signal belongs. This classification can be achieved by the use of the discriminant functions:

gᵢ(x)=ln p(x|ωᵢ)+ln P(ωᵢ) (7)

which, assuming that the densities p(x|ωᵢ) are multivariate normal, becomes:

$${g}_{i} ( x ) = - \frac{1}{2} {( x - {μ}_{i} )}^{t} {E}_{i}^{- 1} ( x - {μ}_{i} ) - \frac{d}{2} ln 2 π - \frac{1}{2} ln \vert {E}_{i} \vert - ln P ( {ω}_{i} ) \qquad ( 8 )$$

To classify the vibroarthrogram as either arthritic or healthy, the prior probabilities for both categories in this embodiment may be assumed to be equal, e.g.,

$$P ( {ω}_{1} ) = P ( {ω}_{2} ) = \frac{1}{2} \qquad ( 9 )$$

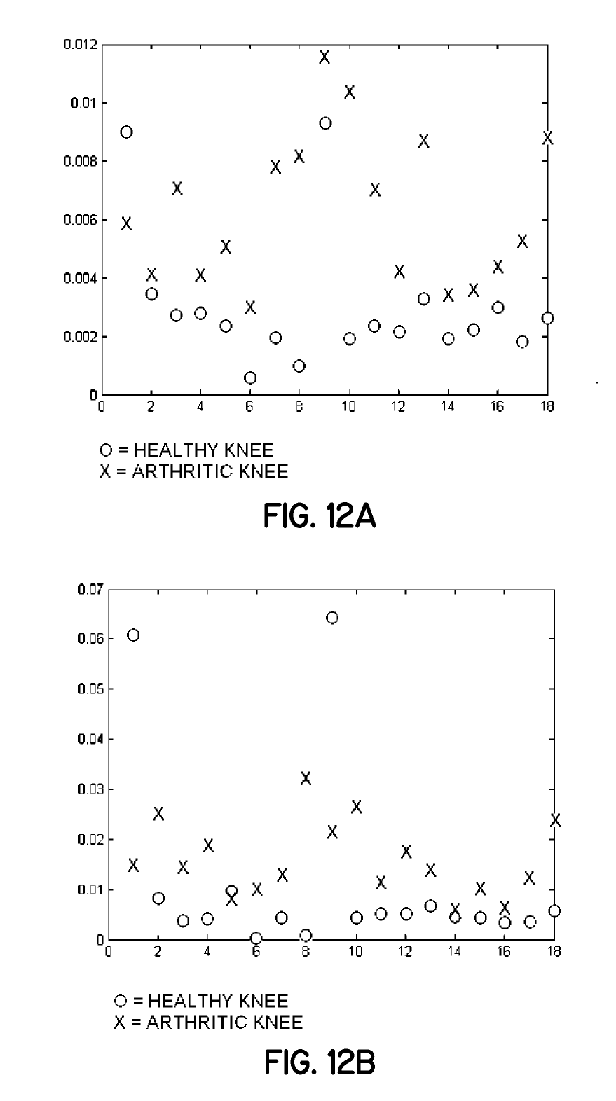

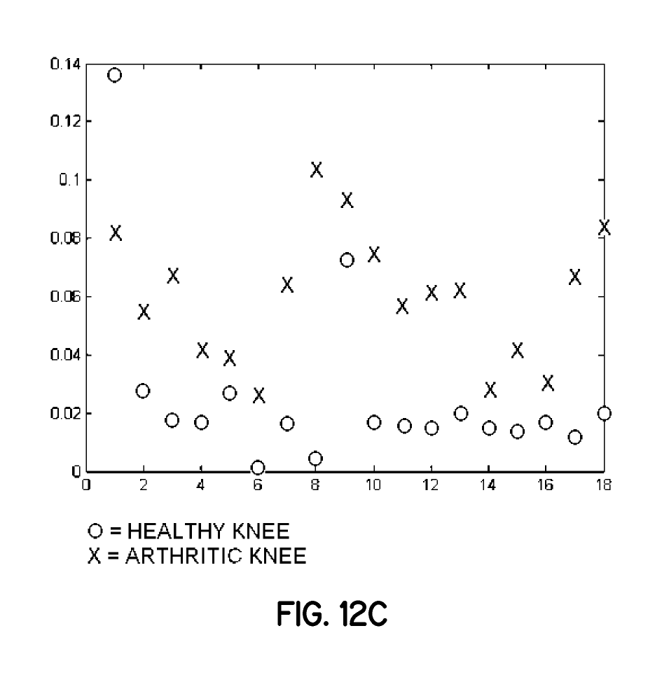

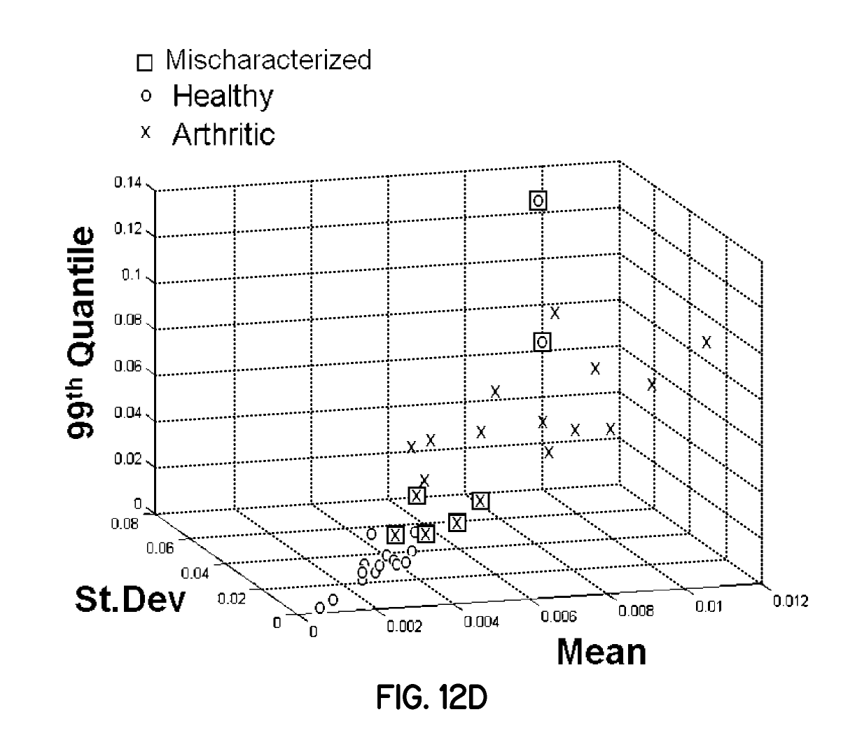

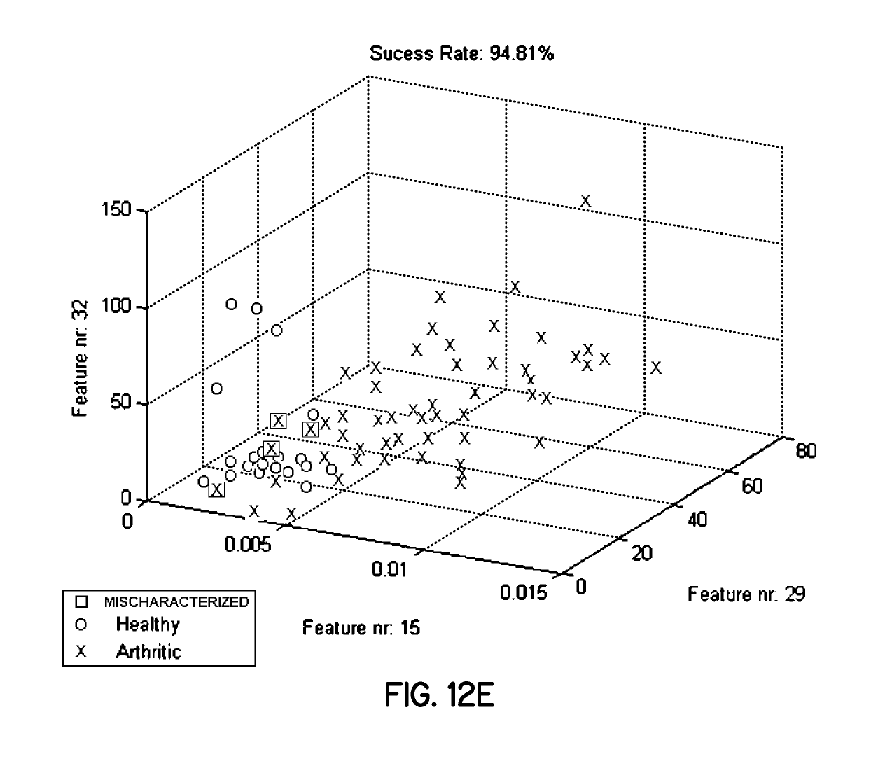

Referring now to FIG. 12A, a scatter plot is presented showing mean values of vibroarthrograms for 18 healthy knees and 18 arthritic knees. As can be seen, the rectified vibroarthrograms for the arthritic knees tend to have higher mean values than the vibroarthrograms for the healthy knees. Thus, in an embodiment of the invention, the mean of the rectified signal may be used as a single feature to design a dichotomizer that classifies the vibroarthrograms. For example, drawing a horizontal line across the plot at a mean of approximately 0.003 provides reasonable separation of the arthritic vibroarthrograms from the healthy vibroarthrograms. Experimental results indicate that the expected success rate obtained using the mean of the rectified vibroarthrogram as a this single feature classifier is about 73%.

Referring now to FIG. 12B, a scatter plot is presented showing standard deviation values of vibroarthrograms for the 18 healthy knees and 18 arthritic knees. As can be seen, the arthritic knees also tend to have higher standard deviation values than the healthy knees. In an alternative embodiment of the invention in which the, a second feature, standard deviation, is included in the discriminant function a second feature, experimental. Experimental results indicate that the expected success rate of this alternative embodiment increased to about 81%.

Referring now to FIG. 12C, a scatter plot is presented showing 99th quintile values of vibroarthrograms for the 18 healthy knees and 18 arthritic knees. As can be seen, the arthritic knees also tend to have higher 99th quintile values than the healthy knees, indicating that the 99th quintile may also be a useful feature to improve separation between vibroarthrograms for healthy joints and injured joints.

FIG. 12D presents a 3-D graph showing the results from applying the discriminant function of equation (8) when applied to a group of test subjects including 15 subjects having healthy knees and 20 subjects suffering from arthritis. These patients were recruited from a group of candidates for the primary total knee replacement procedure. The plot shows subjects that were diagnosed as having healthy knees as O's and those diagnosed as having arthritic knees as X's. Misdiagnosed subjects have a box around the character. That is, a boxed X is someone diagnosed as having an arthritic knee by the discriminant function but that was found to have a healthy knee during a subsequent total knee replacement procedure. Likewise, a boxed O represents a subject diagnosed as having a healthy knee that was later determined to have an arthritic knee. As can be seen, the correct diagnosis was returned in 28 out of the 35 cases, for a success rate of about 80%. FIG. 12E presents a 3-D graph showing results obtained using a discriminant function including 33 features. In this embodiment, the success rate was about 95%. Thus, proper selection and weighting of statistical features of vibroarthrograms can provide accurate diagnoses of joint conditions in a non-invasive manner that does not require exposing the patient to ionizing radiation.

In an embodiment of the invention, the feature vectors 206n-206m may be provided to the diagnosis and visualization module 142 and be included the feature vector set 146. This may enable the diagnosis and visualization module 142 to utilize other feature vectors provided by the ultrasound module 52 and inertial tracking module 48 along with feature vectors 206n-206m to form a complete feature vector set 146. This feature vector set 147 may then be used by the classification and diagnosis modules 147, 148 to provide a diagnosis.

In an embodiment of the invention, Fourier transforms 187 may be used to process vibration data. A Fourier transform is a mathematical operation that transforms a signal from the time domain to the frequency domain. The Fourier transform views the frequency content of an interval of the signal as a whole. That is, it looks at the time interval being transformed, and converts that portion of the signal into the frequency domain so that the frequency content of the interval is revealed. Thus, Fourier transforms switch the dimension of time with the dimension of frequency for the analyzed interval.

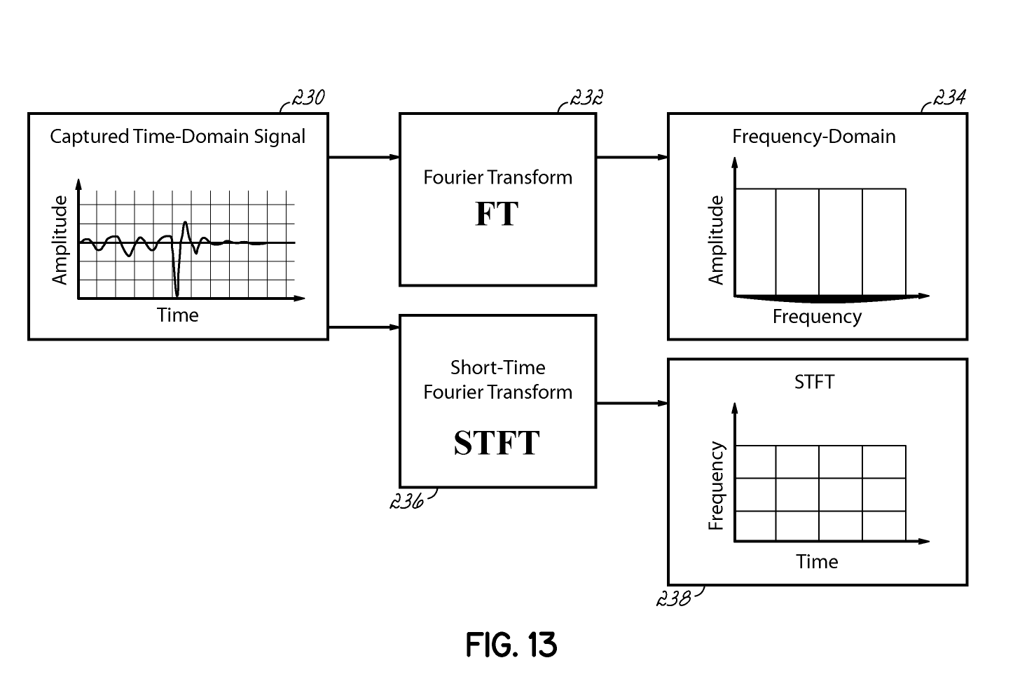

Referring to FIG. 13, the vibration content of the signals from accelerometers 168-171 may be filtered to remove the motion component of the vibration and an interval of the filtered signal defined. This defined interval may provide a captured time-domain signal 230 that is provided to a Fourier transform module 232. The Fourier transform module 232 may use a Discrete Fourier Transform (DFT) to convert the captured time-domain signal 230 from the time to the frequency domain. The resulting captured frequency-domain signal 234 may then be used to analyze the propertied of the captured time-domain signal 230. A DFT is a Fourier transform applied to a sampled (i.e., discrete) signal rather than a continuous signals. DFT's are thus well adapted for processing digital signals. A Fast Fourier Transform (FFT) is a simplified version of the DFT that may be applied to digitized intervals or sample periods when the number of samples in the sample period is a power of two. An FFT computation takes approximately N*log²(N) operations, whereas a DFT takes approximately N² operations, so the FFT is significantly faster.

As a result of applying a Fourier transform to convert the captured time domain signal 230 to the captured-frequency domain signal 234, the time information is obscured. However, the vibration data collected by the vibration detection module 30 of joint monitoring system 30 is part of a larger set of data (i.e., the position data captured by inertial tracking and ultrasound modules 48, 52) that characterizes the joint. Because this additional data is in the time-domain, it may be difficult to correlate a specific characteristic the captured-frequency domain signal 234 with an occurrence of a particular event identified by the time-domain signals generated by inertial tracking and ultrasound modules 48, 52. The vibration signals collected by the vibration detection module 50 may also contain numerous non-stationary or transitory characteristics, i.e., abrupt changes, beginnings and ends of events. These characteristics may be a valuable part of the signal since they may indicate the occurrence of certain events of interest. However, these events are typically not identified or detected by a Fourier analysis of the time-domain signal.

In order to address this short coming of Fourier transforms, the captured time domain signal 230 may be processed by a short-time Fourier transform module 236, which use a windowing technique to analyze the vibration signals and output a short-time frequency domain signal 238. To this end, the short-term Fourier transform module applies a Fourier transform over several small sections of the signal so that both time and frequency information is captured. This type of analysis is called the short-time Fourier Transform (STFT) analysis.

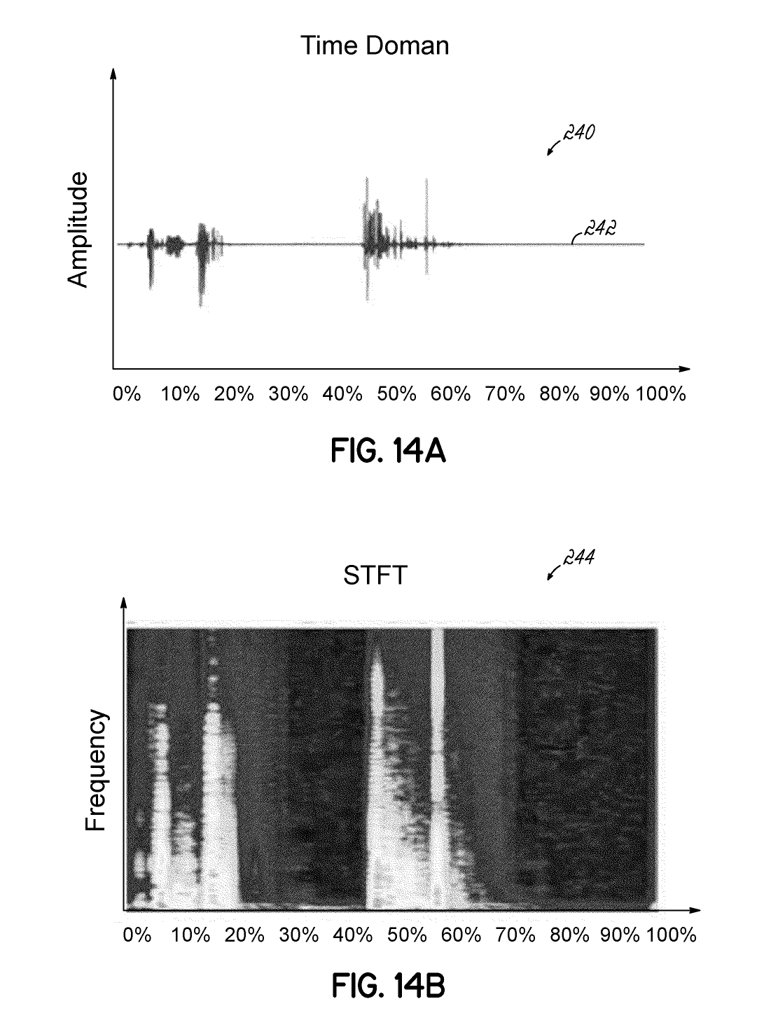

Referring now to FIGS. 14A and 14B, a graph 240 illustrates a exemplary captured time domain signal 242. A corresponding spectrogram 246 may be generated from the time-domain signal 240 using an STFT. The spectrogram 244 reveals the portion of the captured time domain signal's energy with respect to frequency as the captured time domain signal 240 changes over time. Since the spectrogram 244 reveals both time information and information about the frequency content of the signal 242, the spectrogram 242 may be used to filter and analyze vibration signals in both time and frequency simultaneously. A feature vector containing the frequency of a vibration signal occurring at a particular time period may then be employed as an input to the radial basis function network 204 for further analysis or input as part of feature vector set provided to the diagnosis and visualization module 142 by the feature vector module 146.

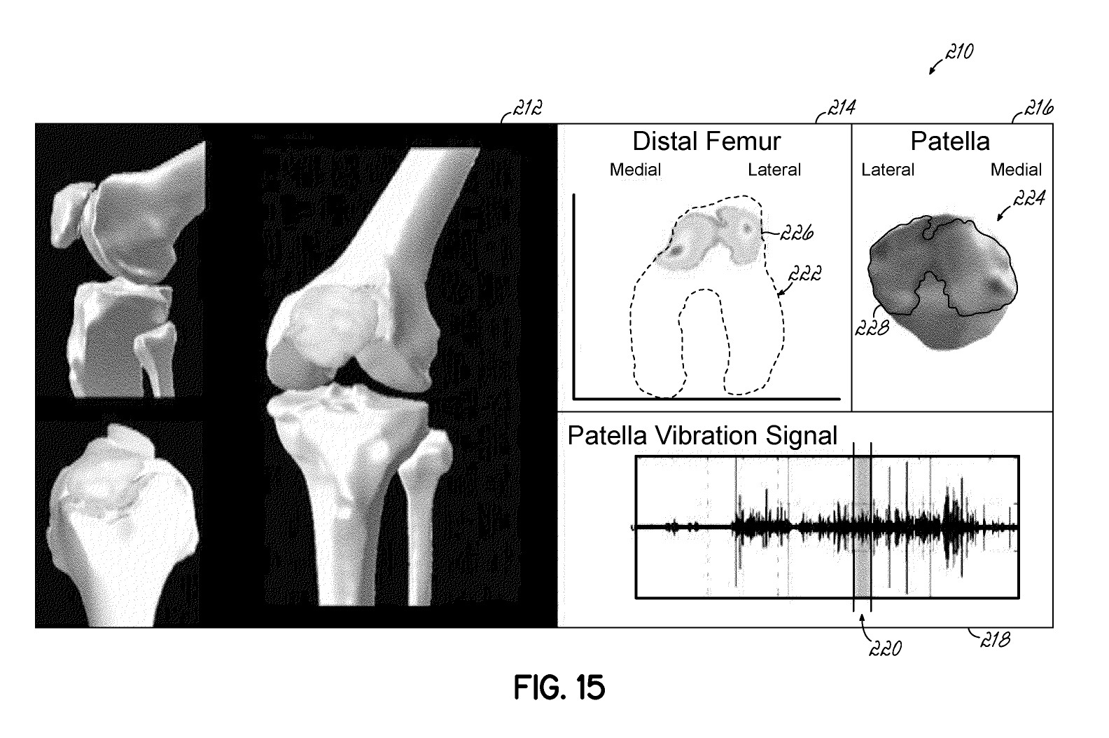

Referring now to FIG. 15, the diagnostic system 140 may be configured to display diagnostic information page 210 that includes an image 212 representing one or more views of the 3-D model of the patient's joint, contact location maps of the distal femur 214 and patella 216, and a vibroarthrogram 218 including a sampling window 220. The contact location maps 214, 216 may include an end view 222 of the distal femur, and an end view 224 of the patella. At different positions of the joint model, respective contact locations 226, 228 may change in size and color to indicate areas of pressure or contact between the bones of the joint. This diagnosis can be extended to include the tibio-femoral joint interaction. The image 212, contact location maps 214, 216 and vibroarthrogram 218 may also be synchronized with respect to joint position. The contact location maps 214, 216 may also include visual feedback of the condition of the joint based on surgical observations entered into the database 130 from one or more medical evaluation sheets 150. The visually observed condition of the joint may thereby be correlated with respect to the vibration signal in the sample window 220.

Upon playback, this synchronization may cause the sampling window 220 to move across the vibroarthrogram 218 in synchronization with movement of the 3-D model 212 and changes to the contact location maps 214, 216. The system user may also be able to examine specific positions of the joint by dragging the sample window 220 across the vibroarthrogram 218. The diagnostic information page 210 may thereby provide a “movie” that allows the system user to simultaneously observe the kinematics of the joint (as shown by the motion of the 3-D model in image 212), with the corresponding contact between the joint components, and the joint vibrations being produced. The in-vivo kinematics and predicted contact location map correlated with the vibration data may bring insight into not only the severity of the articular cartilage degeneration, but also to other defects as determined by the cartilage and meniscus damage map generated based on the surgical observations entered into the database 130. The location of the contact between the interacting surfaces, when co-related with the joint condition of the corresponding location may provide insight in to the nature of the vibration signal in sample window 220 that co-relates to a certain defect. The contact location maps 214, 216 may provide information on the location of the contact between two bones. The grid based visual representation of the evaluation sheet may provide information on the condition of the joint at the location predicted by the contact location maps 214, 216. And the vibration signal in the sampling window 220 may provide information on the exact vibration pattern corresponding to the degenerative condition of the joint being analyzed. Thus, the diagnostic information page 210 may provide the joint information in a unique and comprehensive manner that is invaluable for treatment planning.

The above described vibroarthrography based diagnostic system may be used for pre-operative diagnosis to help determine a proper course of treatment. The system is also well adapted for use perioperatively, and could be used, for example, to assist surgeons with fine tuning a joint implant to ensure that the implant is properly sized and positioned within the patient prior to closing the joint. Moreover, the system may also be useful for post-operative evaluation of implant wear and for early identification of abnormal wear or conditions. The information provided by the diagnostic system may also provide users with immediate feedback on the effectiveness of joint injections (e.g., Visco supplementation) and other treatments. Thus, the diagnostic system may be helpful for surgeons planning joint replacement procedures, and may eliminate the need for imaging modalities involving harmful radiation.

While the invention has been illustrated by a description of various embodiments, and while these embodiments have been described in considerable detail, it is not the intention of the applicant to restrict or in any way limit the scope of the appended claims to such detail. Additional advantages and modifications will readily appear to those skilled in the art. Accordingly, departures may be made from such details without departing from the spirit or scope of applicant's general inventive concept.

What is claimed is:

1. A method of determining a condition of a joint, the method comprising:
receiving a first signal in a processor, the first signal indicative of a vibration generated by a motion of the joint;
generating a vibroarthrograph from the first signal;
extracting a first signal feature from the vibroarthrograph based on a first statistical parameter of the vibroarthrograph;
comparing the first signal feature to a plurality of signal features in a database, each of the plurality of signal features in the database being associated with at least one joint condition; and
determining the condition of the joint based at least in part on a correspondence between the first signal feature and a signal feature of the plurality of signal features in the database.

2. The method of claim 1 further comprising:
receiving a second signal in the processor, the second signal indicative of a position of the joint during the motion of the joint;
synchronizing the first and second signals so that each point on the vibroarthrograph is associated with a position of the joint;
comparing the generated vibroarthrograph with a plurality of vibroarthrographs stored in the database, the comparison being based at least in part on a position based correlation between the generated vibroarthrograph and each of the stored vibroarthrographs, wherein
determining the condition of the joint includes selecting a joint condition in the database based at least in part on a level of position based correlation between the generated vibroarthrograph and a vibroarthrograph of the plurality of vibroarthrographs in the database.

3. The method of claim 1 wherein the first signal feature is one of a plurality of signal features extracted from the vibroarthrograph, the method further comprising:
defining a feature vector of the vibroarthrograph based on two or more signal features of the plurality of signal features;
comparing the defined feature vector with a plurality of feature vectors, each feature vector of the plurality of feature vectors being associated with at least one of the joint conditions stored in the database; and
determining the condition of the joint based at least in part on the comparison between the defined feature vector and the feature vectors stored in the database.

4. The method of claim 1 wherein the first signal feature is extracted from the vibroarthrograph based on a statistical parameter selected from a group of statistical parameters consisting of a complexity, a skewness, a kurtosis, an entropy, a mean, a standard deviation, a signal envelope integral, and a quantile.

5. The method of claim 1 further comprising:
generating a vibroarthrogram for each of a plurality of test subject joints from a group of healthy test subjects;
generating a vibroarthrogram for each of a plurality of test subjects joints from a group of test subjects suffering from an abnormal joint condition; and
associating each of the vibroarthrograms with the condition of the respective test subject's joint in the database.

6. The method of claim 5 further comprising:
receiving data in the database regarding an observed condition of a test subject's joint; and
associating the received data with the vibroarthrogram of the test subject's joint in the database.

7. The method of claim 6 wherein the observed condition is a one of a condition of the cartilage of the test subject's joint, a condition of a ligament of the test subject's joint, or a condition of a meniscus of the test subject's joint.

8. The method of claim 7 wherein the observed condition is given a numerical value rating based on a set of preselected conditions.

9. The method of claim 5 further comprising:
extracting signal features from the test subject vibroarthrograms for each of the pluralities of test subjects; and
associating each of the extracted signal features with the condition of the corresponding joint, wherein
the plurality of signal features in the database includes the signal features extracted from the test subject vibroarthrograms.

10. The method of claim 1 wherein the condition of the joint is determined pre-operatively, perioperatively, or post-operatively.

11. A method of determining a condition of a joint, the method comprising:
receiving a first signal in a processor, the first signal indicative of a vibration generated by a motion of the joint;
generating a vibroarthrograph from the first signal;
receiving a second signal in the processor, the second signal indicative of a position of the joint during the motion of the joint;
determining an orientation of a 3-D model of the joint based at least in part on the second signal;
synchronizing the first and second signals so that each point on the vibroarthrograph is associated with a position of the joint;
displaying a first image representing the orientation of the 3-D model of the joint; and
displaying a second image representing the vibroarthrograph, wherein
the first and second images are synchronized so that movement of the 3-D model corresponds to a position of a sample window in the vibroarthrograph.

12. The method of claim 11 wherein the joint includes a first bone and a second bone, the method further comprising:
determining a contact area between the first bone and the second bone; and
displaying a third image representing the contact area between the first and second bones, the third image being synchronized with the first and second images so that the displayed contact area corresponds to the displayed position of the 3-D model and the sample window.

13. The method of claim 12 wherein the contact area between the first and second bones is determined based on the position of the 3-D joint model.

14. The method of claim 12 further comprising:
overlaying a visual map of a degenerated condition on the contact area between the first and second bones so that the third image includes the visual map.

15. A method of determining a condition of a joint, the method comprising:
receiving a first signal in a processor, the first signal indicative of a vibration generated by a motion of the joint;
generating a vibroarthrograph based on the first signal;
extracting a plurality of signal features from the vibroarthrograph, each signal feature being based on a different statistical parameter;
defining a plurality of feature vectors of the vibroarthrograph, each feature vector being based on one or more weighted signal features of the plurality of signal features and being associated with at least one joint condition;
determining a score for each of the plurality of feature vectors based on the vibroarthrograph; and
diagnosing the joint by selecting a joint condition associated with the feature vector having the highest score.

16. The method of claim 15 further comprising:
receiving a second signal in the processor, the second signal indicative of a position of the joint during the motion of the joint;
synchronizing the first and second signals so that each point on the vibroarthrograph is associated with a position of the joint;
comparing the generated vibroarthrograph with a plurality of vibroarthrographs stored in the database, the comparison being based at least in part on a position based correlation between the generated vibroarthrograph and each of the stored vibroarthrographs, wherein
determining the condition of the joint includes selecting a joint condition in the database based at least in part on a level of position based correlation between the generated vibroarthrograph and a vibroarthrograph of the plurality of vibroarthrographs in the database.

17. A system for determining a condition of a joint, the system comprising:
a processor; and
a memory including program code that, when executed by the processor, causes the processor to:
receive a first signal indicative of a vibration generated by a motion of the joint;
generate a vibroarthrograph from the first signal;
extract a first signal feature from the vibroarthrograph based on a first statistical parameter of the vibroarthrograph;
compare the first signal feature to a plurality of signal features in a database, each of the plurality of signal features in the database being associated with at least one joint condition; and
determine the condition of the joint based at least in part on a correspondence between the first signal feature and a signal feature of the plurality of signal features in the database.

18. A system for determining a condition of a joint, the system comprising:
a processor; and
a memory including program code that, when executed by the processor, causes the processor to:
receive a first signal indicative of a vibration generated by a motion of the joint;
generate a vibroarthrograph from the first signal;
receive a second signal in the processor, the second signal indicative of a position of the joint during the motion of the joint;
determine an orientation of a 3-D model of the joint based at least in part on the second signal;
synchronize the first and second signals so that each point on the vibroarthrograph is associated with a position of the joint;
display a first image representing the orientation of the 3-D model of the joint; and
display a second image representing the vibroarthrograph, wherein
the first and second images are synchronized so that movement of the 3-D model corresponds to a position of a sample window in the vibroarthrograph.

19. A system for determining a condition of a joint, the system comprising:
a processor; and
a memory including program code that, when executed by the processor, causes the processor to:
receive a first signal in a processor, the first signal indicative of a vibration generated by a motion of the joint;
generate a vibroarthrograph based on the first signal;
extract a plurality of signal features from the vibroarthrograph, each signal feature being based on a different statistical parameter;
define a plurality of feature vectors of the vibroarthrograph, each feature vector being based on one or more weighted signal features of the plurality of signal features and being associated with at least one joint condition;
determine a score for each of the plurality of feature vectors based on the vibroarthrograph; and
diagnose the joint by selecting a joint condition associated with the feature vector having the highest score.
